SEQUENCE LISTING (4 sequences)

SEQ ID NO: 1 - protein, 288 aa, Human immunodeficiency virus type 1
FLDGIDKAQEEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNFKRKGGIGGYSAGERIVDIIATDIQTKELQKQITKIQNFRVYYRDSRDPVWKGPAKLLWKGEGAVVIQDNSDIKVVPRRKAKIIRDYGKQMAGDDCVASRQDED

SEQ ID NO: 2 - DNA, 20 bp, Artificial
  Synthetic oligonucleotide primer
tgtgtgcccgtctgttgtgt

SEQ ID NO: 3 - DNA, 20 bp, Artificial
  Synthetic oligonucleotide primer
gagtcctgcgtcgagagagc

SEQ ID NO: 4 - protein, 84 aa, Artificial
  Synthetic amino acid sequence of Figure 1
NFKRKGGIGGYSAGERIVDIIATDIQTKELQKQITKIQNFRVYYRDSRDPVWKGPAKLLWKGEGAVVIQDNSDIKVVPRRKAKI

(19) United States
(12) Patent Application Publication
(10) Pub. No.: US 2016/0230190 A1
(43) Pub. Date: Aug. 11, 2016

(54) Lentiviral Vectors Having a Mutated Integrase Protein and uses Thereof

(71) Applicants: INSERM ( INSTITUT NATIONAL DE LA SANTE ET DE LA RE CHERCHE MEDICALE), Paris (FR); CEA, Gif-sur-Yvette (FR); UNIVERSITE PARIS SUD, Orsay (FR)

(72) Inventors: Che Serguera, Le Kremlin Bicetre (FR); Francois Lachapelle, Le Kremlin Bicetre (FR); Noelle Dufour, Le Kremlin Bicetre (FR); Muhammad Qamar Saeed, Le Kremlin Bicetre (LE)

(21) Appl. No.: 15/022,722

(22) PCT Filed: Sep. 17, 2014
(86) PCT No.: PCT/EP2014/069799

§ 371 Date: Mar. 17, 2016

(30) Foreign Application Priority Data
Sep. 17, 2013 (EP) 13306269.5

(51) Int. Cl. C12N 15/86; A61K 39/00; C12N 15/64; C12N 9/12; C12N 7/00

(52) U.S. Cl. CPC C12N 15/86; C12N 9/1241; C12N 7/00; C12N 15/64; A61K 39/00; A61K 48/00

(57) ABSTRACT

The present invention relates to a lentiviral vector wherein the expressed integrase protein comprises at least one point mutation consisting of the substitution of the aspartic acid residue at position 167 by an amino acid selected from the group consisting of histidine, arginine and lysine.

### FIELD OF THE INVENTION

The present invention relates to lentiviral vectors having a mutated integrase protein and uses thereof.

### BACKGROUND OF THE INVENTION

Amongst a wide variety of viral vectors developed for gene transfer, those based on HIV-1 are highly prized for their efficiency and compliance. HIV-1, a human lentivirus causing AIDS is one of the most studied microorganisms ever. This favored the early development [1] and improvement [2] of HIV-1 non-replicating vectors, which are able to transduce cells at any stage of mitosis. Reverse transcription of the viral RNA genome into a double strand DNA and provirus integration into the cell chromatin are characteristic events of all retroviruses including HIV-1 and derived vectors. These events are mediated by the viral enzymes reverse transcriptase (RT) and integrase (IN) through coordinated interactions with cellular factors [3]. Within capsids as part of the reverse-transcription complex and then in the pre-integration complex (PIC), IN ability to interact with RT, form homo-oligomers, bind to the viral DNA and complex with other viral and cellular proteins, is central for effective infection [3, 4].

Biochemical activity of IN consists in two reactions modifying the viral DNA, i) the 3′ processing of LTR extremities, and the pairing of the processed LTR termini to chromosomal DNA [5]. In addition, IN ability for sequential multimerization is mandatory for proper interaction with viral and cellular factors involved in viral processing and integration [4, 6]. The proteins Lens epithelium-derived growth factor (LEDGF/p75) and the karyopherin transportin 3 (TNPO3) are two cellular factors that interact with IN and facilitate integration of the provirus [7-9].

For instance, after completion of reverse transcription, IN forms dimers at provirus ends and then interact with LEDGF/p75 inducing the formation of a IN tetramer enclosing processed extremities of the provirus [4]. LEDGF/p75 then guides the PIC towards transcribed regions of chromatin where IN catalyzes provirus insertion preferentially within expressed genes [6, 10]. TNPO3 also interacts with IN and favors integration within gene rich regions [7-9].

During HIV infection only about half of all provirus having reached the nucleus integrate while the rest circularize and remain as episomes [17]. Such rate of nuclear circles is increased when integration is impaired, by modifying IN integrity, its substrate the viral LTRs, or reducing the availability of its cellular cofactors LEDGF/p75 [15, 18-20]. Among these strategies, using IN mutants is the best way to prevent HIV DNA integration [20, 21]. Few mutations on this protein only preclude integration (class I mutations) while other mutations produce a deeper phenotype and affect as well other steps of the virus or vector processing (class II mutations) [22].

HIV-1 IN is constituted by 3 functional domains, a zinc binding N-terminal domain (NTD), a Mg2+ consuming central catalytic domain (CCD), and a C-terminal domain (CTD) that also binds DNA [22, 23]. Typical class I mutations are those modifying any of the 3 amino acids DDE (D64, D116 and E152) of the catalytic triad [22]. Of these mutations, the D64V substitution is the most used in studies with NILV [20, 21]. Other mutations along the 3 IN domains with mild class II phenotype might generate different vector attributes of particular interest for gene transfer.

### SUMMARY OF THE INVENTION

The present invention relates to a lentiviral vector wherein the expressed integrase protein comprises at least one point mutation consisting of the substitution of the aspartic acid residue at position 167 by a histidine.

### DETAILED DESCRIPTION OF THE INVENTION

HIV-1 derived vectors are among most efficient for gene transduction in mammalian tissues. As the parent virus, they carry out vector genome insertion into the host cellular chromatin. But because not random, their pattern of integration rises several conceptual and safety issues. To solve part of these questions, HIV-derived vectors have been engineered to be non-integrating. This was mainly achieved by mutating HIV-1 integrase at functional hotspots of the enzyme enabling the development of streamlined functional episomes for transgene expression. Few integrase mutant vectors have been successfully tested so far for gene transfer. They are cleared with time in mitotic cells but stable within non-dividing retina cells or neurons. Here the inventors characterized several HIV-vectors carrying mutant integrases known to modify viral enzyme activity, oligomerization or interaction with key cellular cofactor of provirus integration as p75/LEDGF or TNPO3. They show that these mutations differently affected transduction efficiencies as well as rates and patterns of integration of HIV-derived vectors. Most interestingly, they report that one of these mutant (D167H) vectors improves transduction efficiency and integration in both 293T and primary CD34+ cells.

Accordingly a first object of the present invention relates to a lentiviral vector wherein the expressed integrase protein comprises at least one point mutation consisting of the substitution of the aspartic acid residue at position 167 by an amino acid selected from the group consisting of histidine, arginine and lysine,

In some embodiments, the present invention relates to a lentiviral vector wherein the expressed integrase protein comprises at least one point mutation consisting of the substitution of the aspartic acid residue at position 167 by a histidine,

Like other retroviruses, lentiviruses possess genes gag, pol and env sequences flanked by two LTR (Long Terminal Repeat). Each of these genes encodes several proteins that are initially expressed as a single precursor polypeptide. The gag gene encodes the internal structural proteins (capsid and nucleocapsid). The pol gene encodes the reverse transcriptase, integrase and protease. The env gene encodes the viral envelope glycoprotein. The genome of lentiviruses element further contains a RRE (Rev responsive element) cis-acting responsible for the export from the nucleus of the viral RNA genome to be packaged. LTR sequences 5 and 3′ are used to promote transcription and polyadenylation of viral RNA. The LTR contains all other cis-acting sequences necessary for viral replication. Sequences necessary for reverse transcription of the genome (the binding site of the tRNA primer) and the encapsidation of viral RNA into particles (site Ψ) are adjacent to the 5′LTR. If the sequences necessary for encapsidation (or packaging of retroviral RNA into infectious virions) are missing from the viral genome, genomic RNA will not be actively packaged. The lentiviral genome also includes accessory genes such as vif, vpr, vpu, nef, TAT, REV, etc.

As used herein the expression “lentiviral vector” has its general meaning in the art and encompasses a lentiviral vector particle that comprises both proteins and genetic material, preferably encapsidated into these proteins. Particles are made of viral envelope proteins (encoded by an env gene) as well as structural proteins (encoded by a gag gene). Inside the particles, a viral core (or capsid) contains three enzymes (encoded by a pol gene), i.e., the reverse transcriptase, the integrase and the protease, and genetic material (lentiviral genome).

As used herein, the term “Integrase (IN)” encompasses the IN protein mediates the insertion of the lentivirus (e.g. HIV-1) proviral DNA into the genomic DNA of an infected cell. This process is mediated by three distinct functions of IN. First, an exonuclease activity trims two nucleotides from each 3′ end of the linear viral DNA duplex. Then, a double-stranded endonuclease activity cleaves the host DNA at the integration site. Finally, a ligase activity generates a single covalent linkage at each end of the proviral DNA. An exemplary amino acid sequence for the integrase of HIV-1 is provided by SEQ ID NO:1.

SEQ ID NO: 1
FLDGIDKAQEEHEKYHSNWRAMASDFNLPPVVAKEIVASCDKCQLKGEAM
HGQVDCSPGIWQLDCTHLEGKVILVAVHVASGYIEAEVIPAETGQETAYF
LLKLAGRWPVKTVHTDNGSNFTSTTVKAACWWAGIKQEFGIPYNPQSQGV
IESMNKELKKIIGQVRDQAEHLKTAVQMAVFIHNFKRKGGIGGYSAGERI
VDIIATDIQTKELQKQITKIQNFRVYYRDSRDPVWKGPAKLLWKGEGAVV
IQDNSDIKVVPRRKAKIIRDYGKQMAGDDCVASRQDED

In some embodiments, the lentiviral vector of the invention is selected from the group consisting of HIV-1, HIV-2, SIV, FIV, EIAV, BIV, VISNA and CAEV vectors.

In a particular embodiment, the lentiviral vector is a HIV-1 vector.

In a particular embodiment, the lentiviral vector of the invention is a recombinant lentivirus non-replicative and non-integrative, that is to say, it is incapable of autonomous replication and specific integration of the transduced cells. Accordingly, in some embodiments, the integrase protein is devoid of the capacity of integration of the lentiviral genome into the genome of the host cells i.e., the integrase protein is mutated to specifically alter its integrase activity. Accordingly the integrase capacity of the protein is altered whereas the correct expression of the GAG, PRO and POL proteins and/or the formation of the capsid and hence of the vector particles, as well as other steps of the viral cycle, preceding or subsequent to the integration step, such as the reverse transcription, the nucleus import, stay intact.

In a particular embodiment of the invention, the property of the integrase of being defective, results from a mutation of class 1, preferably amino acid substitutions (one-amino acid substitution) or short deletions giving rise to a protein fulfilling the requirements of the preceding paragraph. The mutation is carried out within the pol gene. Examples of mutations altering HIV-1 and enabling to obtain a non-functional integrase for integration (integration-incompetent integrase) are the following: H12N, H12C, H16C, H16V, S81R, D41A, K42A, H51A, Q53C, D55V, D64E, D64V, E69A, K71A, E85A, E87A, D116N, D116I, D116A, N120G, N120I, N120E, E152G, E152A, D35E, K156E, K156A, E157A, K159E, K159A, K160A, R166A, E170A, H171A, K173A, K186Q, K186T, K188T, E198A, R199C, R199T, R199A, D202A, K211A, Q214L, Q216L, Q221 L, W235F, W235E, K236S, K236A, K246A, G247W, D253A, R262A, R263A and K264H. Another proposed substitution is the replacement of the amino acids residues RRK (positions 262 to 264) by the amino acids residues AAH.

In some embodiments, the envelope protein of the lentiviral vector of the invention may be pseudotyped with the envelope protein of the lentivirus used to prepare the lentiviral vector, or alternatively with a heterogeneous envelope protein that is chosen with respect to the cells to be targeted into the host.

In a particular embodiment, said lentiviral vector is pseudotyped with a VSV-G protein. The VSV-G glycoprotein may originate from different serotypes of the genus of the vesiculoviruses: VSV-Indiana serotype, VSV-New Jersey serotype or other glycoproteins of the vesiculoviruses such as Piry, Chandipura, Isfahan and Cocal. The VSV-G glycoprotein is chosen among species classified in the vesiculovirus genus: Carajas virus (CJSV), Chandipura virus (CHPV), Cocal virus (COCV), Isfahan virus (ISFV), Maraba virus (MARAV), Piry virus (PIRYV), Vesicular stomatitis Alagoas virus (VSAV), Vesicular stomatitis Indiana virus (VSIV) and Vesicular stomatitis New Jersey virus (VSNJV) and/or Grass carp rhabdovirus, BeAn 157575 virus (BeAn 157575), Boteke virus (BTKV), Calchaqui virus (CQIV), Eel virus American (EVA), Gray Lodge virus (GLOV), Jurona virus (JURV), Klamath virus (KLAV), Kwatta virus (KWAV), La Joya virus (LJV), Malpais Spring virus (MSPV), Mount Elgon bat virus (MEBV), Perinet virus (PERV), Pike fry rhabdovirus (PFRV), Porton virus (PORV), Radi virus (RADIV), Spring viremia of carp virus (SVCV), Tupaia virus (TUPV), Ulcerative disease rhabdovirus (UDRV) and Yug Bogdanovac virus (YBV).

In a particular embodiment, the VSV-G protein originating from a VSV is modified with respect to its native form, especially to improve pseudotyping.

In a particular embodiment, the envelope protein comprises domains or fragments originating from different envelope protein(s) of different viruses, especially of different genus of different species of VSV. In the case of VSV, the G protein comprises or consists of the transmembrane domain of the indiana VSV and the ectodomain of a strain of a different VSV serotype. In a particular embodiment, the envelope protein(s) comprises the transmembrane domain of the indiana VSV and the ectodomain of the New-Jersey VSV.

In another aspect, the lentiviral vector of the invention is pseudotyped with HA protein (influenza-hemaglutinin), RD114 protein, modified envelopes with inserted cell-specific ligands or with viral envelope proteins originated from a virus selected in one or several of the following orders or families: Arenaviridae, Flaviridae, Togaviridae, Coronaviridae, Orthomyxoviridae, Retroviridae and Mononegavirales including Paramyxoviridae, Rhabdoviridae or Filoviridae. Any virus envelope protein is suitable for pseudotyping, assuming that the pseudotyping is compatible with production and purification steps of lentiviral vector particles.

The structure and composition of the vector genome used to prepare the lentiviral vectors of the invention are in accordance with those described in the art. Especially, minimum lentiviral gene delivery vectors can be prepared from a vector genome, which only contains, apart from the heterologous polynucleotide(s) of interest (i.e. the transgene(s)), the sequences of the lentiviral genome which are non-coding regions of said genome, necessary to provide recognition signals for DNA or RNA synthesis and processing. Hence, a vector genome may be a replacement vector in which all the viral coding sequences between the 2 long terminal repeats (LTRs) have been replaced by the polynucleotide(s) of interest (i.e. the transgene).

In a particular embodiment the lentiviral vector genome also comprises in addition, a polynucleotide consisting in the DNA flap. The DNA flap (defined in Zennou V. et al 2000, Cell vol 101, 173-185 or in WO 99/55892 and WO 01/27304), is a structure which is central in the genome of some lentiviruses especially in HIV retroviruses, where it gives rise to a 3-stranded DNA structure normally synthesized during especially HIV reverse transcription and which acts as a cis-determinant of HIV genome nuclear import. The DNA flap enables a central strand displacement event controlled in cis by the central polypurine tract (cPPT) and the central termination sequence (CTS) during reverse transcription. When inserted in lentiviral derived vectors, the polynucleotide enabling the DNA flap to be produced during retro-transcription, stimulates gene transfer efficiency and complements the level of nuclear import to wild-type levels (Arhel N. et al, Retrovirology 2006, 3:38, 26 Jun. 2006, Wild-type and central DNA flap defective HIV-1 lentiviral vector genomes: intracellular visualization at ultra structural resolution levels).

In a particular embodiment, the DNA flap is inserted immediately upstream of the transgene(s), advantageously to have a central position in the vector genome. A DNA flap suitable for the invention may be obtained from a retrovirus, especially from a lentivirus, or from a retrovirus-like organism such as retrotransposon, either prepared synthetically (chemical synthesis) or by amplification of the DNA flap from any retrovirus especially from a lentivirus nucleic acid such as by Polymerase chain reaction (PCR). The DNA flap may be obtained from a retrovirus, especially a lentivirus, especially from a human retrovirus or lentivirus and in particular a HIV retrovirus, or from the CAEV (Caprine Arthritis Encephalitis Virus) virus, the EIAV (Equine Infectious Anaemia Virus) virus, the VISNA virus, the SIV (Simian Immunodeficiency Virus) virus or the FIV (Feline Immunodeficiency Virus) virus.

In a particular embodiment, the DNA flap is obtained from an HIV retrovirus, for example HIV-1 or HIV-2 virus including any isolate of these two types. It is noteworthy that the DNA flap is used as a DNA fragment isolated from its natural (viral genome) nucleotide context i.e., out of the context of the pol gene in which it is naturally contained in the lentivirus genome. Therefore, the DNA flap is used, in the present invention, deleted from the unnecessary 5′ and 3′ parts of the pol gene and is recombined with sequences of different origin. The DNA flap may be either prepared synthetically (chemical synthesis) or by amplification of the DNA providing the DNA flap from the appropriate source as defined above such as by Polymerase chain reaction (PCR).

A particular appropriate polynucleotide comprising the structure providing the DNA flap is a 178-base pair polymerase chain reaction (PCR) fragment encompassing the cPPT and CTS regions of the HIV-1 DNA

The lentiviral vector genome may also comprise regulatory signals for transcription and expression of non lentiviral origin, such as a promoter and/or an enhancer. Examples of promoters that can be used in immune response elicitation are CMV also referred to as CMVie (CMV immediate early), EF1α promoter, CGA promoter, CD11c promoter and house keeping gene promoters such as PGK promoter, ubiquitin promoter, actin promoter, histone promoter, alpha-tubulin promoter, beta-tubulin promoter, superoxide dismutase 1 (SOD-1) promoter, dihydrofolate reductase (DHFR) promoter, hypoxanthine phosphorybosyltransferase (HPRT) promoter, adenosine deaminase promoter, thymidylate synthetase promoter, dihydrofolate reductase P1 promoter, glucose-6-phosphate sehydrogenase promoter or nucleolin promoter.

In a particular embodiment, the transgene is under the control of regulatory signals for transcription and expression.

In some embodiment, the lentiviral vector genome comprises all the elements necessary for the nucleic import and the correct expression of the polynucleotide of interest (i.e. the transgene). As examples of elements that can be inserted in the lentiviral genome of the lentiviral vector of the invention are at least one (preferably two) long terminal repeats (LTR), such as a LTR5′ and a LTR3′, a psi sequence involved in the lentiviral genome encapsidation, and optionally at least one DNA flap comprising a cPPT and a CTS domains.

In a particular embodiment of the invention, the LTR, preferably the LTR3′, is deleted for the promoter and the enhancer of U3; this modification has been shown to increase substantially the transcription of the transgene inserted in the lentiviral genome (WO01/27304).

In some embodiments, the lentiviral vector genome may also comprise elements selected among a splice donor site (SD), a splice acceptor site (SA) and/or a Rev-responsive element (RRE).

In some embodiments, the lentiviral vector genome is devoid of functional gag, pol and/or env lentiviral genes. By “functional” it is meant a gene that is correctly transcribed, and/or correctly expressed. Thus, the lentiviral vector genome of the invention in this embodiment contains at least one of the gag, pol and env genes that is either not transcribed or incompletely transcribed; the expression “incompletely transcribed” refers to the alteration in the transcripts gag, gag-pro or gag-pro-pol, one of these or several of these being not transcribed. In a particular embodiment, the lentiviral genome is devoid of gag, pol and/or env lentiviral genes.

In a particular embodiment the lentiviral vector genome is also devoid of the coding sequences for Vif-, Vpr-, Vpu- and Nef-accessory genes (for HIV-1 lentiviral vectors), or of their complete or functional genes.

The lentiviral vector of the invention is non replicative i.e., the vector and lentiviral vector genome are not able to form new particles budding from the infected host cell. This may be achieved by the absence in the lentiviral genome of the gag, pol or env genes, as indicated in the above paragraph; this can also be achieved by deleting other viral coding sequence(s) and/or cis-acting genetic elements needed for particles formation.

In some embodiments, the lentiviral vector of the invention comprises (i) a recombinant genome comprising, between the LTR sequences 5 ‘and 3’ lentiviral, a psi sequence lentiviral packaging, a nuclear export element RNA, at least one transgene and, optionally, a promoter and/or a sequence favoring the nuclear import of the RNA, and (ii) a mutated integrase protein according to the invention.

In some embodiments, the recombinant genome comprises for the sequence 5 ‘LTR-psi-RRE-cPPT-CTS-transgene(s)-3’ LTR.

In some embodiments, the recombinant genome comprises the sequence 5 ‘LTR-psi-RRE-cPPT-CTS-promoter-transgene(s)-3’ LTR.

As used herein, the term “transgene” or “polynucleotide of interest” refers to any nucleic acid that shall be expressed in a mammal cell. Typically the nucleic acid is a coding or non coding nucleic acid. It can be a non-coding sequence such as for example a recognition sequence of an enzyme (site specific integration, site with a particular affinity for a protein, etc.). This is preferably a sequence encoding a given polypeptide or RNA active as such. It may include a cDNA, a gDNA, a synthetic DNA, an RNA, for example, a siRNA, a ribozyme, etc. or a combination thereof. Typically, the transgene is a DNA comprising a sequence encoding the desired expression product. The transgene may also include one or more regions of transcription termination, typically a polyadenylation signal.

In some embodiments, the transgene may be selected from a nucleic acid catalyst (interfering, antisense, ribozyme), a nucleic acid suicide (eg, encoding a toxin) or a nucleic acid encoding a biologically active peptide, such as a growth factor, a trophic factor, one anti-angiogenic factor, a hormone, a cytokine, an antibody, a receptor, a differentiation factor, a colony stimulating factor, an anticancer agent, an enzyme, a neurotransmitter or its precursor, etc. According to a particular embodiment of the invention, the transgene encodes eg trophic factors include: RdCVF, CNTF, NGF, NT3, NT4, FGF, PDGF, GDNF, etc., Or for anti-angiogenic factors or enzymes restaurant deficient metabolic activity or providing a particular metabolic function, for example: TH, AADC, GTPC, β-glucuronidase, etc.

According to another particular embodiment of the invention, the transgene encodes, for example, RNA interference (RNAi) to inhibit specifically the expression of mutated proteins involved in a disease or a dominant genetic disease caused by a gain of function, such as a neurodegenerative disease such as mutated SOD (Amyotrophic Lateral Sclerosis), protein APP, tau, presenilin, or BACE (Alzheimer's disease), the α-synuclein (Parkinson's disease) or Huntingtin (Huntington disease).

In certain circumstances, the transgene encodes for a site-specific endonuclease that provides for site-specific knock-down of gene function, e.g., where the endonuclease knocks out an allele associated with a retinal disease. For example, where a dominant allele encodes a defective copy of a gene that, when wild-type, is a retinal structural protein and/or provides for normal retinal function, a site-specific endonuclease (such as TALEnucleases, meganucleases or Zinc finger nucleases) can be targeted to the defective allele and knock out the defective allele. In addition to knocking out a defective allele, a site-specific nuclease can also be used to stimulate homologous recombination with a donor DNA that encodes a functional copy of the protein encoded by the defective allele. Thus, e.g., the lentiviral vector of the invention can be used to deliver both a site-specific endonuclease that knocks out a defective allele, and can be used to deliver a functional copy of the defective allele, resulting in repair of the defective allele, thereby providing for production of a functional protein.

In some embodiments, the transgene encodes for an antigenic polypeptide. A polypeptide is said antigenic when its sequence contains at least one peptide (epitope) able to elicit an immune response when put in contact with antigen presenting cells (APC). Typically, the antigenic polypeptide comprises at least one B epitope, capable of eliciting a humoral immune response, particularly a protective humoral response, or a T-epitope capable of eliciting a cellular immune response. In a particular embodiment, the at least one polypeptide is encoded by a nucleotide sequence originating from the genome of a pathogen, such as a virus, especially a retrovirus, lentivirus, flavivirus or coronavirus, of a bacterium or of a parasite. In another embodiment, the antigenic polypeptide of the invention comprises or consists in surface antigens, such as viral envelope or other membrane proteins, and fragments thereof, for example envelope from AIDS viruses, including HIV-1 or HIV-2, or for example envelope from the Yellow Fever Virus, the West Nile Virus, the Dengue virus (DV), the Japanese encephalitis virus (JEV) or the SARS-associated coronavirus. Other interesting viral polypeptides are from the capsid of HIV. Alternatively, the antigenic polypeptide is derived from a tumoral antigen or a tumoral epitope. Particular polypeptides (or part thereof) are those expressed on the cell surface of tumoral cells. These polypeptides (or part thereof) may originate from the cell (self peptide) either in a wild type or mutated form; they also may originate from a virus that transforms a normal cell in tumor cell (tumor virus). Examples of such viruses, etiologic agents for human cancer, are the Human Papilloma Virus (HPV) causing cervical cancer, the Epstein-Barr Virus causing lymphoma through the EBV-induced membrane antigen (EBMA), HTLV-1 causing Acute T cell leukaemia (ACT) through the HTLV-1 tax protein, the human herpes virus type 8 (HHV8), the hepatitis B virus (HBV) and the hepatitis C virus (HCV).

The transgene is typically placed under the control of a transcriptional promoter, which may be homologous to the transgene- or heterologous promoter such as a cellular, viral, synthetic, chimeric, etc. The promoter used may be constitutive or regulated, weak or strong, tissue-specific or ubiquitous dependent RNA polymerase 2 or 3, etc. It typically uses a viral promoter such as CMV, RSV LTR, TK, etc. or preferably a cellular promoter such as PGK, Rho, EF1α, etc. Of tissue-specific promoters can be used. It may be, for example promoters ENO, GFAP, NSE, a promoter of RNA polymerase III promoter such as U6 or H1, possibly modified, etc. The promoter used to drive expression of the transgene can be for example a viral promoter selected from the gene promoter CMV, RSV LTR or TK.

The lentiviral vectors of the invention can be produced by any well-known method in the art including by transfection (s) transient (s), in stable cell lines and/or by means of helper virus.

Typically, the lentiviral vector of the invention is obtainable by a transcomplementation system (vector/packaging system) by transfecting in vitro a permissive cell (such as 293T cells) with a plasmid containing the lentiviral vector genome of the invention, and at least one other plasmid providing, in trans, the gag, pol and env sequences encoding the polypeptides GAG, POL and the envelope protein(s), or for a portion of these polypeptides sufficient to enable formation of retroviral particles.

As an example, permissive cells are transfected with a) transcomplementation plasmid, lacking packaging signal psi and comprising a sequence lentiviral gag and pol sequence encoding an integrase mutated according to the invention, the plasmid is optionally deleted of accessory genes vif, nef, vpu and/or vpr, b) a second plasmid (envelope expression plasmid or pseudotyping env plasmid) comprising a gene encoding an envelope protein(s) (such as VSV-G) and c) a plasmid vector comprising a recombinant genome lentiviral, optionally deleted from the promoter region of the 3 ‘LTR or U3 enhancer sequence of the 3’ LTR, including, between the LTR sequences 5 ‘and 3’ lentiviral, a psi encapsidation sequence, a nuclear export element (preferably RRE element of HIV or other retroviruses equivalent), the transgene and optionally a promoter and/or a nuclear import sequence (cPPT sequence eg CTS) of the RNA.

Advantageously, the three plasmids used do not contain homologous sequence sufficient for recombination. Nucleic acids encoding gag, pol and env cDNA can be advantageously prepared according to conventional techniques, from viral gene sequences available in the prior art and databases.

The trans-complementation plasmid provides a nucleic acid encoding the proteins lentiviral gag and pol. These proteins are derived from a lentivirus, and most preferably, from HIV-1. The plasmid is devoid of encapsidation sequence, sequence coding for an envelope, accessory genes, and advantageously also lacks lentiviral LTRs. Therefore, the sequences coding for gag and pol proteins are advantageously placed under the control of a heterologous promoter, eg cellular, viral, etc., which can be constitutive or regulated, weak or strong. It is preferably a plasmid containing a sequence transcomplémentant Δpsi-CMV-gag-pol-PolyA. This plasmid allows the expression of all the proteins necessary for the formation of empty virions, except the envelope glycoproteins. The plasmid transcomplementation may advantageously comprise the TAT and REV genes. Plasmid transcomplementation is advantageously devoid of vif, vpr, vpu and/or nef accessory genes. It is understood that the gag and pol genes and genes TAT and REV can also be carried by different plasmids, possibly separated. In this case, several plasmids are used transcomplementation, each encoding one or more of said proteins.

The promoters used in the plasmid transcomplementation, the envelope plasmid and the plasmid vector respectively to promote the expression of gag and pol of the coat protein, the mRNA of the vector genome and the transgene are promoters identical or different, chosen advantageously from ubiquitous promoters or specific, for example, from viral promoters CMV, TK, RSV LTR promoter and the RNA polymerase III promoter such as U6 or H1 or promoters of helper viruses encoding env, gag and pol (i.e. adenoviral, baculoviral, herpes viruses).

For the production of the lentiviral vector of the invention, the plasmids described above can be introduced into competent cells and viruses produced are harvested. The cells used may be any cell competent, particularly eukaryotic cells, in particular mammalian, eg human or animal. They can be somatic or embryonic stem or differentiated. Typically the cells include 293T cells, fibroblast cells, hepatocytes, muscle cells (skeletal, cardiac, smooth, blood vessel, etc.), nerve cells (neurons, glial cells, astrocytes) of epithelial cells, renal, ocular etc. It may also include, insect, plant cells, yeast, or prokaryotic cells. It can also be cells transformed by the SV40 T antigen.

The genes gag, pol and env encoded in plasmids or helper viruses can be introduced into cells by any method known in the art, suitable for cell type considered. Usually, the cells and the vector system are contacted in a suitable device (plate, dish, tube, pouch, etc. . . . ), for a period of time sufficient to allow the transfer of the vector system or the plasmid in the cells. Typically, the vector system or the plasmid is introduced into the cells by calcium phosphate precipitation, electroporation, transduction or by using one of transfection-facilitating compounds, such as lipids, polymers, liposomes and peptides, etc. The calcium phosphate precipitation is preferred. The cells are cultured in any suitable medium such as RPMI, DMEM, a specific medium to a culture in the absence of fetal calf serum, etc.

Once transfected the lentiviral vectors of the invention may be purified from the supernatant of the cells. Purification of the lentiviral vector to enhance the concentration can be accomplished by any suitable method, such as by density gradient purification (e.g., cesium chloride (CsCl)) or by chromatography techniques (e.g., column or batch chromatography). For example, the vector of the invention can be subjected to two or three CsCl density gradient purification steps. The vector, is desirably purified from cells infected using a method that comprises lysing cells infected with adenovirus, applying the lysate to a chromatography resin, eluting the adenovirus from the chromatography resin, and collecting a fraction containing the lentiviral vector of the invention.

The lentiviral vector according to the invention can be used for expressing the transgene in a mammal cell of interest, more particularly in cells that do not divide or the transient expression of the transgene in division cells that are refractory to other methods of transfection or transduction. The lentiviral vectors of the invention are able to transduce various cell types such as, for example, liver cells (e.g. hepatocytes), muscle cells, brain cells, kidney cells, retinal cells, and hematopoietic cells. In some embodiments, the target cells of the present invention are “non-dividing” cells. These cells include cells such as neuronal cells that do not normally divide. However, it is not intended that the present invention be limited to non-dividing cells (including, but not limited to muscle cells, white blood cells, spleen cells, liver cells, eye cells, epithelial cells, etc.).

Possible applications of lentiviral vectors of the invention are of several types and include gene therapy, ie, the gene transfer in any mammal cell, in particular in human cells. It may be dividing cells or quiescent cells, cells belonging to the central organs or peripheral organs such as the liver, pancreas, muscle, heart, etc. This is preferably a gene transfer into quiescent cells (which do not divide), Gene therapy may allow the expression of proteins, eg neurotrophic factors, enzymes, transcription factors, receptors, etc. It also enables to implement a strategy “oligonucleotide” (interfering RNA or antisense, ribozymes, etc.) cell therapy, ie, the expression of differentiation factors in progenitor cells to guide cell fate to a selected before transplantation or ex vivo transduction of cells to express an interest factor, followed by transplantation of the said cells.

Lentiviral vectors according to the invention may also particularly suitable for research purposes.

Lentiviral vectors according to the invention may also be particularly suitable for the production of vaccines or for eliciting a vaccine response in a subject in need thereof.

The lentiviral vector according to the invention may also be used as a medicament. The lentiviral vector according to the invention may be particularly suitable for treating a disease in a subject.

In some embodiments, the lentiviral vector of the invention can be used to treat a cancer. Non-limiting examples of cancers that can be treated according to the invention include breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyo sarcoma, Kaposi's sarcoma, polycythemia vera, essential thrombocytosis, Hodgkin's disease, non-Hodgkin's lymphoma, soft-tissue sarcoma, mesothelioma, osteogenic sarcoma, primary macroglobulinemia, and retinoblastoma, and the like.

In some embodiments, the lentiviral vector of the invention can be used to treat an autoimmune disorder including, but not limited to, a disorder selected from the group consisting of Achlorhydra Autoimmune Active Chronic Hepatitis, Acute Disseminated Encephalomyelitis, Acute hemorrhagic leukoencephalitis, Addison's Disease, gammaglobulinemia, Agammaglobulinemia, Alopecia areata, Amyotrophic Lateral Sclerosis, Ankylosing Spondylitis, Anti-GBM/TBM Nephritis, Antiphospholipid syndrome, Antisynthetase syndrome, Arthritis, Atopic allergy, Atopic Dermatitis, Aplastic Anemia, Autoimmune cardiomyopathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome Types I, H, & III, Autoimmune progesterone dermatitis, Autoimmune thrombocytopenic purpura, Autoimmune uveitis, Balo disease/Balo concentric sclerosis, Bechets Syndrome, Berger's disease, Bickerstaff s encephalitis, Blau syndrome, Bullous Pemphigoid, Castleman's disease, Chronic Fatigue Immune Dysfunction Syndrome, chronic inflammatory demyelinating polyneuropathy, Chronic recurrent multifocal ostomyelitis, Churg-Strauss syndrome, Cicatricial Pemphigoid, Coeliac Disease, Cogan syndrome, Cold agglutinin disease, Complement component 2 deficiency, Cranial arteritis, CREST syndrome, Crohns Disease, Cushings Syndrome, Cutaneous leukocytoclastic angiitis, Degos disease, Dermatitis herpetiformis, Dermatomyositis, Diabetes mellitus type 1, Diffuse cutaneous systemic sclerosis, Dressler's syndrome, Discoid lupus er thematosus, eczema, Enthesitis-related arthritis, Eosinophilic fasciitis, Epidermolysis bullosa acquisita, Erythema nodosum, Essential mixed cryoglobulinemia, Evan's syndrome, Fibrodysplasia ossificans progressiva, Fibromyositis, Fibrosing aveolitis, Gastritis, Gastrointestinal pemphigoid. Giant cell arteritis, Goodpasture's syndrome, Graves' disease. Guillain-Barre syndrome (GBS), Hashimoto's encephalitis, Hashimoto's thyroiditis, Hemolytic anaemia, Henoc-Schonlein purpura, Herpes gestationis, Hughes syndrome (or Antiphospholipid syndrome). Hypogammaglobulinemia, Idiopathic Inflammatory Demyelinating Diseases, Idiopathic pulmonary fibrosis, Idiopathic thrombocytopenic purpura, IgA nephropathy (or Bergefs disease), Inclusion body myositis, ory demyelinating polyneuopathy, Juvenile idiopathic arthritis, Juvenile rheumatoid arthritis, Lambert-Eaton myasthenic syndrome, Leukocytoclastic vasculitis, Lichen planus, Lichen sclerosus, Linear IgA disease (LAD), Lou Gehrig's Disease, Lupoid hepatitis, Lupus erythematosus, Majeed syndrome, Meniere's disease, Microscopic polyangiitis, Miller-Fisher syndrome, Mixed Connective Tissue Disease, Mucha-Habermann disease, Muckle-Wells syndrome, Multiple Myeloma, Myasthenia gravis, Myositis, Narcolepsy, Neuromyelitis optica (also Devic's Disease), Occular cicatricial pemphigoid, Ord thyroiditis, Palindromic rheumatism, PANDAS (Pediatric Autoimmune Neuropsychiatric Disorders Associated with Streptococcus), Paraneoplastic cerebellar degeneration, Paraneoplastic cerebellar degeneration, Parry Romberg syndrome, Parsonnage-Turner syndrome, Pars planitis, Pemphigus, Pemphigus vulgaris, Pernicious anaemia, Perivenous encephalomyelitis, POEMS syndrome, Polyarteritis nodosa, Polymyalgia rheumatica, Polymyositis, Primary biliary cirrhosis, psoriasis, psoriatic arthritis, Pyoderma gangrenosum, pure red cell aplasia, Rasmussen's encephalitis, Raynaud phenomenon, Relapsing polychondritis, Reiter's syndrome, Retroperitoneal fibrosis, Rheumatoid arthritis, Rheumatoid fever, Schmidt syndrome, Schnitzler syndrome, Scleritis, Sjogren's syndrome, Spondyloarthropathy, sticky blood syndrome, Still's Disease, Subacute bacterial endocarditis (SBE), Susac's syndrome, Sweet syndrome, Sydenham Chorea, Sympathetic ophthalmia, Takayasu's arteritis, Temporal arteritis, Tolosa-Hunt syndrome, Transverse Myelitis, Ulcerative Colitis, Undifferentiated connective tissue disease, Undifferentiated spondyloarthropathy, vasculitis, Wegener's granulomatosis, Wilson's syndrome, and Wiskott-Aldrich syndrome.

In some embodiments, the lentiviral vector of the invention can be used to treat an ocular disorder that includes, but is not limited to, a disorder selected from the group consisting of glaucoma including Open Angle Glaucoma (e.g., Primary Open Angle Glaucoma, Pigmentary Glaucoma, and Exfoliative Glaucoma, Low Tension Glaucoma), Angle Closure Glaucoma (also known clinically as closed angle glaucoma, narrow angle glaucoma, pupillary block glaucoma, and ciliary block glaucoma) (e.g., Acute Angle Closure Glaucoma and Chronic Angle Closure Glaucoma), Aniridic Glaucoma, Congenital Glaucoma, Juvenile Glaucoma, Lens-Induced Glaucoma, Neovascular Glaucoma (e.g., using vectors composed of Vascular Endothelial Growth Factor (VEGF) decoy, Pigment Derived Growth Factor (PDGF), Endostatin, Angiostatin, or Angiopoetin-1), Post-Traumatic Glaucoma, Steroid-Induced Glaucoma, Sturge-Weber Syndrome Glaucoma, and Uveitis-Induced Glaucoma, diabetic retinopathy (e.g., using vectors composed of VEGF decoy, PDGF, Endostatin, Angiostatin, or Angiopoetin-1), macular degeneration (e.g. vectors composed of VEGF decoy, PDGF, Endostatin, Angiostatin, Angiopoetin-1, ATP Binding Casette Subfamily A Member 4), macular degeneration (e.g., using vectors composed of VEGF decoy, PDGF, Endostatin, Angiostatin, Angiopoetin-1, ATP Binding Casette Subfamily A Member 4), choroidal neovascularization, (e.g., using vectors composed of VEGF decoy, PDGF, Endostatin, Angiostatin, or Angiopoetin-1), vascular leak, and/or retinal edema, bacterial conjunctivitis, fungal conjunctivitis, viral conjunctivitis, uveitis, keratic precipitates, macular edema (e.g., using vectors composed of VEGF decoy, PDGF, Endostatin, Angiostatin, or Angiopoetin-1), inflammation response after intra-ocular lens implantation, uveitis syndromes (for example, chronic iridocyclitis or chronic endophthalmitis), retinal vasculitis (for example, as seen in rheumatoid arthritis, juvenile rheumatoid arthritis, systemic lupus erythematosus, progressive systemic sclerosis, polyarteritis nodosa, Wegener's granulomatosis, termporal arteritis, Adamantiades Bechcet disease, Sjorgen's, relapsing polychondritis and HLA-B27 associated spondylitis), sarcoidosis, Eales disease, acute retinal necrosis, Vogt Koyanaki Harada syndrome, occular toxoplasmosis, radiation retinopathy, proliferative vitreoretinopathy, endophthalmitis, ocular glaucomas (for example, inflammatory glaucomas), optic neuritis, ischemic optic neuropathy (e.g. vectors composed of Allotopic NADH dehydrogenase Unit 4), thyroid associated orbitopathy, orbital pseudotumor, pigment dispersion syndrome (pigmentary glaucoma), scleritis, episcleritis choroidopathies (for example, “White-dot” syndromes including, but not limited to, acute multifocal posterior placoid), retinopathies (for example, cystoid macular edema, central serous choroidopathy and presumed ocular histoplasmosis syndrome (e.g., vectors composed of Glial Cell Derived Neurotropic Factor, Peripherin-2)), retinal vascular disease (for example, diabetic retinopathy, Coat's disease and retinal arterial macroaneurysm), retinal artery occlusions, retinal vein occlusions, retinopathy of prematurity, retinitis pigmentosa (e.g. vectors composed of Retinal Pigment Specific 65 kDa protein), familial exudative vitreoretinopathy (FEVR), idiopathic polypoidal choroidal vasculopathy, epiretinal macular membranes and cataracts.

In some embodiments, the lentiviral vector of the invention can be used to treat a blood disorder that includes, but is not limited to, a blood disorder selected from the group consisting of anemia, bleeding and clotting disorders (e.g., disseminated intravascular coagulation (DiC), hemophilia, Henoch-Schonlien Purpura, hereditary hemorrhagic telangiectasia, thrombocytopenia (ITP, TTP), thrombophilia, Von Willebrand's disease), leukemias (e.g., acute lymphocytic leukemia, acute myelocytic leukemia, chronic lymphocytic leukemia, chronic myelocytic leukemia), lymphomas (e.g., Hodgkin lymphoma, non-Hodgkin lymphoma), myeloproliferative disorders (e.g., myelofibrosis, Polycythemia Vera, thrombocythemia), plasma cell disorders (e.g., macroglobulinemia, monoclonal gammopathies of undetermined significance, multiple lyeloma), spleen disorders, white blood cell disorders (e.g., basophilic disorder, eosinophilic disorder, lymphocytopenia, monocyte disorders, neutropenia, neutrophillic leukocytosis), thrombosis, deep vein thrombosis (DVT), hemochromatosis, menorrhagia, sickle cell disease, and thalassemia.

In some embodiments, the lentiviral vector of the invention can be used to treat a neurological disorder that includes, but is not limited to, a neurological disorders selected from the group consisting of Gaucher disease, Parkinson's disease, Alzheimer's disease, amyotrophic lateral sclerosis (ALS), multiple sclerosis (MS), Huntington's disease, Fredrich's ataxia, Mild Cognitive Impairment, Cerebral Amyloid Angiopathy, Parkinsonism Disease, Lewy Body Disease, Frontotemporal Dementia (FTD) Multiple System Atrophy (MSA), Progressive Supranuclear Palsy, and movement disorders (including ataxia, cerebral palsy, choreoathetosis, dystonia, Tourette's syndrome, kernicteras) and tremor disorders, and leukodystrophies (including adrenoleukodystrophy, metachromatic leukodystrophy, Canavan disease, Alexander disease, Pelizaeus-Merzbacher disease), neuronal ceroid lipofucsinoses, ataxia telangectasia, Rett Syndrome, alpha.-synucleinopathy (e.g., Lewy Body Disease, Multiple System Atrophy, Hallervorden-Spatz disease, or Frontotemporal Dementia), Niemann-Pick Type C disease (NPCD), spinocerebellar ataxia Type 1, Type 2, and Type 3, and dentatorubral pallidoluysian atrophy (DRLPA).

In some embodiments, the lentiviral vector of the invention can be used to treat a lung disorder that includes, but is not limited to, a lung disorder selected from the group consisting of asthma, atelectasis, bronchitis, COPD (chronic obstructive pulmonary disease), emphysema, Lung cancer, mesothelioma, pneumonia, asbestosis, Aspergilloma, Aspergillosis, Aspergillosis—acute invasive, bronchiectasis, bronchiolitis obliterans organizing pneumonia (BOOP), eosinophilic pneumonia, necrotizing pneumonia, ral effusion, pneumoconiosis, pneumothorax, pulmonary actinomycosis, monary alveolar proteinosis, pulmonary anthrax, pulmonary arteriovenous malformation, pulmonary fibrosis, pulmonary embolus, pulmonary histiocytosis X (eosinophilic granuloma), pulmonary hypertension, pulmonary edema, pulmonary hemorrhage, pulmonary nocardiosis, pulmonary tuberculosis, pulmonary veno-occlusive disease, rheumatoid lung disease, sarcoidosis, radiation fibrosis, hypersensitivity pneumonitis, acute respiratory distress syndrome (ARDS), infant respiratory distress syndrome, idiopathic pulmonary fibrosis, idiopathic interstitial pneumonia, lymphangioleiomyomatosis, pulmonary Langerhans' cell histiocytosis, pulmonary alveolar proteinosis, sinusitis, tonsillitis, otitis media, pharyngitis, laryngitis, Pulmonary hamartoma, pulmonary sequestration, congenital cystic adenomatoid malformation (CCAM), and cystic fibrosis.

In some embodiments, the lentiviral vector of the invention can be used to treat an infectious disease in a human that includes, but is not limited to, an infectious disease selected from the group consisting of fungal diseases such as dermatophytosis (e.g., trichophytosis, ringworm or tinea infections), athletes foot, paronychia, pityriasis versicolor, erythrasma, intertrigo, fungal diaper rash, Candida vulvitis, Candida balanitis, otitis externa, candidiasis (cutaneous and mucocutaneous), chronic mucocandidiasis (e.g. thrush and vaginal candidiasis), cryptococcosis, geotrichosis, trichosporosis, aspergillosis, penicilliosis, fusariosis, zygomycosis, sporotrichosis, chromomycosis, coccidioidomycosis, histoplasmosis, blastomycosis, paracoccidioidomycosis, pseudallescheriosis, mycetoma, mycotic keratitis, otomycosis, pneumocystosis, and fungemia, Acinetobacter infections, Actinomycosis, African sleeping sickness, AIDS (Acquired immune deficiency syndrome), Amebiasis, Anaplasmosis, Anthrax, Arcanobacterium haemolyticum infection, Argentine hemorrhagic fever, Ascariasis, Aspergillosis, atrovirus infection, Babesiosis, Bacillus cereus infection, Bacterial pneumonia, Bacterial vaginosis (BV), Bacteroides infection, Balantidiasis, Baylisascaris infection, BK virus infection, Black piedra, Blastocystis hominis infection, Borrelia infection, Botulism (and Infant botulism), Brazilian hemorrhagic fever, Brucellosis, Burkholderia infection, Buruli ulcer, Calcivirus infection (Norovirus and Sapovirus), Candidiasis, Cat-scratch disease, Cellulitis, Chagas Disease (American trypanosomiasis), Chancroid, Chickenpox, Chlamydia, Cholera, Chromoblastomycosis, Clonorchiasis, Clostridium difficile, Coccidioidomycosis, Colorado tick fever (CTF), Common cold (Acute viral rhinopharyngitis; Acute coryza), Creutzfeldt-Jakob disease (CJD), Cryptococcosis, Cryptosporidiosis, ous larva migrans (CLM), Dengue fever, Dientamoebiasis, Diphtheria, Diphyllobothriasis, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum, Exanthem subitum, Fasciolopsiasis, Fasciolosis, Fatal familial insomnia (FFI), Filariasis, Fusobacterium infection, Gas gangrene (Clostridial myonecrosis), Geotrichosis, Gerstmann-Straussler-Scheinker syndrome (GSS), Giardiasis Glanders, Gnathostomiasis, Gonorrhea, Granuloma inguinale (Donovanosis), Group A streptococcal infection, Group B streptococcal infection, Haemophilus influenzae, Hand, foot and mouth disease (HFMD), Hantavirus Pulmonary Syndrome (HPS) Helicobacter pylori infection, ic-uremic syndrome (HUS), Hemorrhagic fever with renal syndrome (HFRS), Hepatitis A, B, C, D, E, Herpes simplex, Histoplasmosis, Hookworm infection, n bocavirus infection, Human ewingii ehrlichiosis, Human granulocytic anaplasmosis (HGA), Human granulocytic anaplasmosis (HGA), Human monocytic ehrlichiosis, Human papillomavirus (HPV) infection, Human parainfluenza virus infection, Hymenolepiasis, Epstein-Barr Virus Infectious Mononucleosis (Mono), Influenza (flu), Isosporiasis, Kawasaki disease, Keratitis, Kingella kingae infection, Kuru, Lassa fever, Legionellosis (Legionnaires' disease), Legionellosis (Pontiac fever), Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever (MHF), Measles, Melioidosis (Whitmore's disease), Meningitis, Meningococcal disease, Metagonimiasis, Microsporidiosis, Molluscum contagiosum (MC), Mumps, Murine typhus (Endemic typhus), Mycoplasma pneumonia, Mycetoma, Myiasis, Neonatal conjunctivitis (Ophthalmia neonatorum), (New) Variant Creutzfeldt-Jakob disease (vCJD, nvCJD), Nocardiosis, Onchocerciasis (River blindness), Paracoccidioidomycosis (South American blastomycosis), Paragonimiasis, Pasteurellosis, Pediculosis capitis (Head lice), Pediculosis corporis (Body lice), Pediculosis pubis (Pubic lice, Crab lice), Pelvic inflammatory disease (PID), Pertussis (Whooping cough), Plague, Pneumococcal infection, Pneumocystis pneumonia (PCP), Pneumonia, Poliomyelitis, Poliomyelitis, Prevotella infection, mary amoebic meningoencephalitis (PAM), Progressive multifocal leukoencephalopathy, Psittacosis, Q fever, Rabies, Rat-bite fever, Respiratory syncytial virus infection, Rhinosporidiosis, inovirus infection, Rickettsial infection, Rickettsialpox, Rift Valley fever (RVF), Rocky mountain spotted fever (RMSF), Rotavirus infection, Rubella, Salmonellosis, SARS (Severe Acute Respiratory Syndrome), Scabies, Schistosomiasis, Sepsis, Shigellosis (Bacillary dysentery), Shingles (Herpes zoster), Smallpox (Variola), Sporotrichosis, Staphylococcal food poisoning, Staphylococcal infection, Strongyloidiasis, Syphilis, Taeniasis, tanus (Lockjaw), Tinea barbae (Barber's itch), Tinea capitis (Ringworm of the Scalp), Tinea corporis (Ringworm of the Body), Tinea cruris (Jock itch), Tinea manuum (Ringworm of the Hand), Tinea nigra, Tinea unguium (Onychomycosis), Tinea versicolor (Pityriasis versicolor). Toxocariasis (Visceral Larva Migrans (VLM)), Toxoplasmosis, Trichinellosis, Trichomoniasis, Trichuriasis (Whipworm infection), Tuberculosis, Tularemia, Ureaplasma real iicum infection, Venezuelan equine encephalitis, Venezuelan hemorrhagic fever, viral pneumonia. West Nile Fever, White plectra (Tinea blanca), Yersinia pseudotuberculosis infection, Yersiniosis, Yellow fever, and Zygomycosis.

Lentiviral vectors of the invention can be administered to a subject by any route. In some embodiments the lentiviral vector of the invention is administered to the subject parenterally, preferably intravascularly (including intravenously). When administered parenterally, it is preferred that the vectors be given in a pharmaceutical vehicle suitable for injection such as a sterile aqueous solution or dispersion. Following administration, the subject is monitored to detect the expression of the transgene. Dose and duration of treatment is determined individually depending on the condition or disease to be treated. A wide variety of conditions or diseases can be treated based on the gene expression produced by administration of the gene of interest in the vector of the present invention. The dosage of vector delivered using the method of the invention will vary depending on the desired response by the host and the vector used. Generally, it is expected that up to 100-200 μg of DNA or RNA can be administered in a single dosage, although a range of 0.5 mg/kg body weight to 50 mg/kg body weight will be suitable for most applications.

The invention will be further illustrated by the following figures and examples. However, these examples and figures should not be interpreted in any way as limiting the scope of the present invention.

### FIGURES

FIG. 1

Schematic representation of the vector and the different integrases used in our experiments. A) We used a 2 copies self inactivating (SIN) pTrip Vector expressing the tracer GFP and the resistance gene puromycin N-acetyl transferase (pac) under control of promoters CMV and PGK respectively. Only cis-acting sequences of HIV-1 are present in the vector, the long terminal repeat (LTR), the Rev responsive element (RRE), the encapsidation signal Ψ and the central poly purine tract (cPPT). Enhancer from U3 of LTRs is deleted: Self INactivating vector (SIN). B) Representation of the different Integrase domains: N-terminal domain (NTD) spans the first 49 amino acids. Amino acids 50-212 form the catalytic core domain (CCD) and the C-terminal domain (CTD) extends from amino acid 213 to 288. Mutations introduced for our study are indicated highlighted. Aspartate-64 (D), implicated in catalytic activity, is replaced with valine (V). Aspartate-167 (D) or glutamine-168 (Q) involved in interaction with LEDGF are respectively replaced with histidine (H) and alanine (A) in two different constructs. In IN-LQ, lysine-186 (K) is replaced with glutamine (Q) in L-region and glutamine-214 and 216 (Q) are substituted with two leucines (L). Region N is mutated by replacing 262-arginine-arginine-lysine-264 (RRK) with two alanines and a histidine (AAH). (adapted from [15])

FIG. 2

Tittering methods are compared to define, which of viral RNA copies/ul or ng of p24/ul measurements are better correlated to transducing units (TU/ul) values using different stocks of pTrip SIN vector of 2 different sizes and carrying wild type (WT) or mutated (D167H; D64V; Q168A; N; LQ) integrases. Computed data of all vectors stocks indicate that both variables are statistically correlated to TU/ul (vRNAc/ul vs TU/ul p<0.00001 and ng of p24/ul vs TU/ul<0.0005) as assessed through Pearson's correlation. Independent comparison of these variables in each group of vector indicates that viral RNA variation is correlated to that of transducing units (TU) for all types of vectors but one (LQ), while that of p24/ul reaches significances for only 3 vectors type as assessed through Pearson's correlation. Two types of vectors were used in this comparison, a pTrip-CMV-GFP-SIN of 4100 bp and a pTrip-CMV-GFP/PGK-PAC-SIN of 6400 bp.

FIG. 3.

Comparison of GFP expression in 293T cells transduced with same M.O.I. of pTrip-CMV-GFP/PGK-PAC HIV-vectors carrying different IN mutations. A) Using 10 vRNAc per cell the mutant D167H allows to transduce more cells as compared to WT vector. Other mutant vectors all display a non-integrating phenotype with D64V mutant showing the best transducing efficiency 4 days after transduction. Stars represent statistical significance of the difference between WT and D167H at each time point or between D64V and any other non-integrating mutant. B) Mean fluorescence intensity (MFI) is higher in cells transduced with D167H as compared to that transduced with WT, only when the rate of transduced cells is above 30%. C) At higher dose of 100 vRNAc, transduction efficiencies with all vectors are increased. D) Although both WT and D167H can transduce nearly 100% of the cells, MFI is statistically higher with D167H at all time points. Experiments were done at least 3 times in duplicate. Statistics: One way ANOVA and Tuckey pot hoc test; (NS) p>0.05; (*) p<0.05; (**) p<0.01; (***) p<0.001.

FIG. 4

Comparison of integration rate, in 293T cells, of HIV vectors pTrip-CMV-GFP/PGK-PAC carrying integrases with different mutations. A) Cells were incubated with an amount of each different vector allowing to transduce 30% of the cells as assessed through measuring % of GFP expressing cells with facs. After 4 days cells were cultured in presence of puromycin to select integrating events. A) D167H mutant vector allowed more integrating events than WT (p<0.001) and both WT and D167H permitted appearance of more colonies than any of the non-integrating vectors, represented in this graph with Q168A (p<0.001). B) Of all non-integrating vectors, D64V mutant was the least integrating (p<0.001). All experiments were done 4 times in duplicate. Non-transduced cells all died in puromycin medium. Statistics: one way ANOVA and Tuckey's multiple comparison test, NS p>0.05; (*) p<0.005; (**) p<0.01; (***) p<0.001.

FIG. 5

Comparison of efficiency of transduction and integration, of D64V and D64V+D167H mutant vectors pTrip-CMV-GFP/PGK-PAC (100 vRNAc/cell). A) Transduction efficiency of 293T cells is equivalent with both vectors. B) Both vectors display a non-integrating phenotype as GFP expression decreases with time in dividing 293T cells. C) Cells transduced with vectors carrying the IN D167H+D64V display significantly more integrating events as compared to cells transduced with D64V vectors. Experiments were done in triplicate and with 2 different stocks of vectors for each mutant. Non-transduced cells all died in puromycin medium. Statistics: t-Test two tailed, NSp>0.05; (*) p<0.005; (**) p<0.01; (***) p<0.001.

FIG. 6

Kinetics of integration assessed through puromycin resistance and real time QPCR measurement of vectors pTrip PGK-Puro/CMV-Gfp carrying a D167H IN. Puromycin was added 12, 24, 48 or 72 hrs after transduction and integration was rated as the number of colonies puromycin resistant normalized with respective percentage of GFP expression. (a) and (b) 293T cells transduced with vectors carrying a D64V or D64V+D167H IN. (a) Integration rate at each time point after cells transduction with 50 vRNAc or (b) 100 vRNAc. (c) 293T cells transduced with vectors carrying a WT or a D167H IN. Graph shows vectors integration rate at each time point after cells transduction with 25 vRNAc. Non-transduced cells all died in puromycin medium. For each vector, experiments were done in triplicate with 2 different stocks. (d-g) Real time QPCR measurement of total vector DNA, 2 LTR circles, integrated vector DNA and linear vector DNA at different times after transduction. (d) Total vector DNA between 8 hrs and 72 hrs after transduction to assess reverse transcription. (e) Percentage of 2 LTR circles at different times after transduction as an inverse correlate of integration. (f) Integrated vector DNA normalized with total vector DNA. Note that WT vector does not further integrate between 48 h and 72 h while the amount of integrated vector D167H keeps increasing between 48 h and 72 h. (g) Percentage of linear copies of vector DNA. Experiments done in duplicate. Statistics: two way ANOVA and Bonferroni posttest, NSp>0.05; (*) p<0.005; (**) p<0.01; (***) p<0.001.

FIG. 7

Human hematopoietic stem cells CD34+ were transduced with 10 or 100 copies of viral RNA copies of vectors WT or D167H. Transduction efficiency was assessed measuring the % of GFP expressing cells with FACS, 4 days after transduction. With either amount of vectors, mutant D167H is about two times more efficient to transduce CD34+ cells. Experiments were done twice in duplicate. Statistics: t-Test, NSp>0.05; (*) p<0.005; (**) p<0.01; (***) p<0.001.

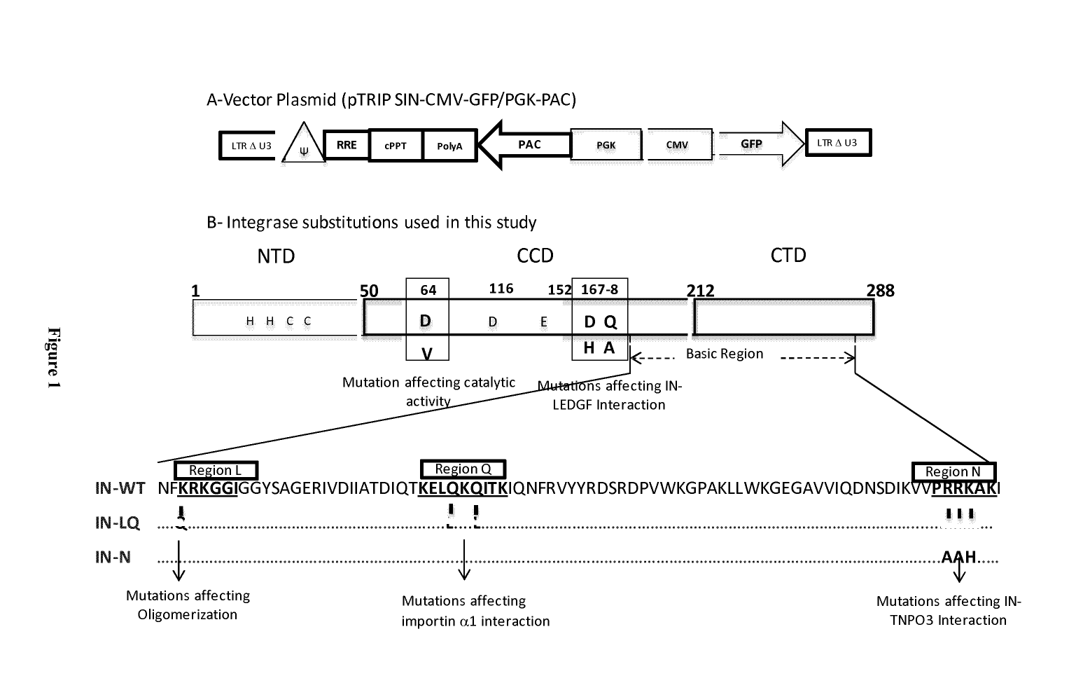

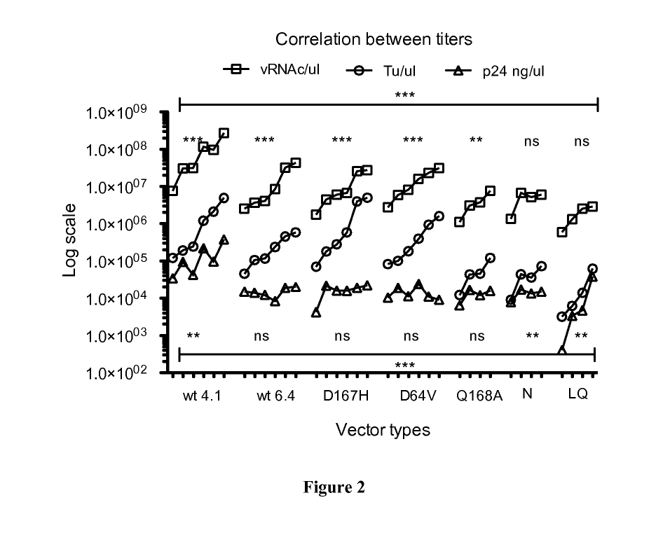

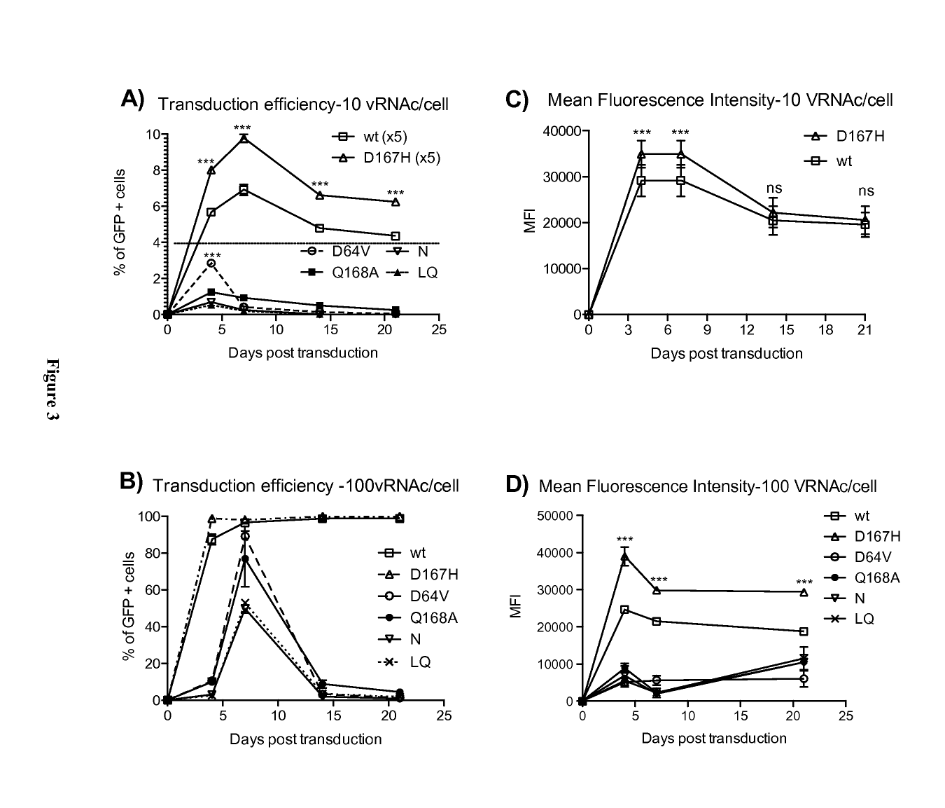

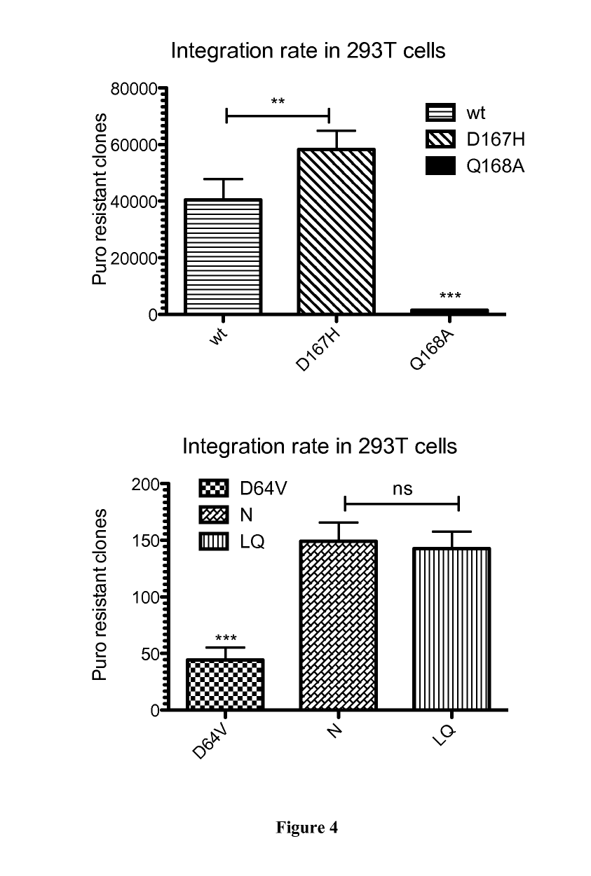

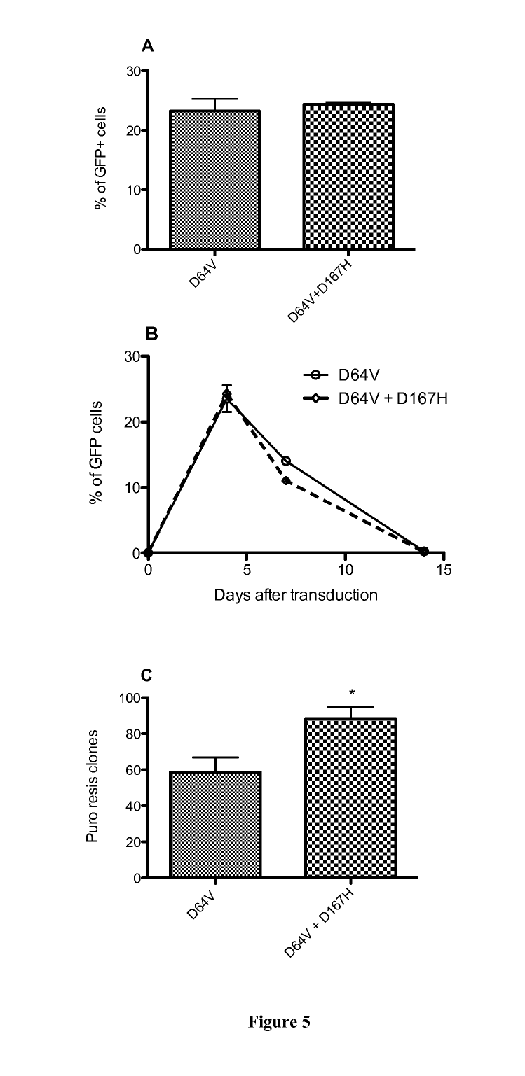

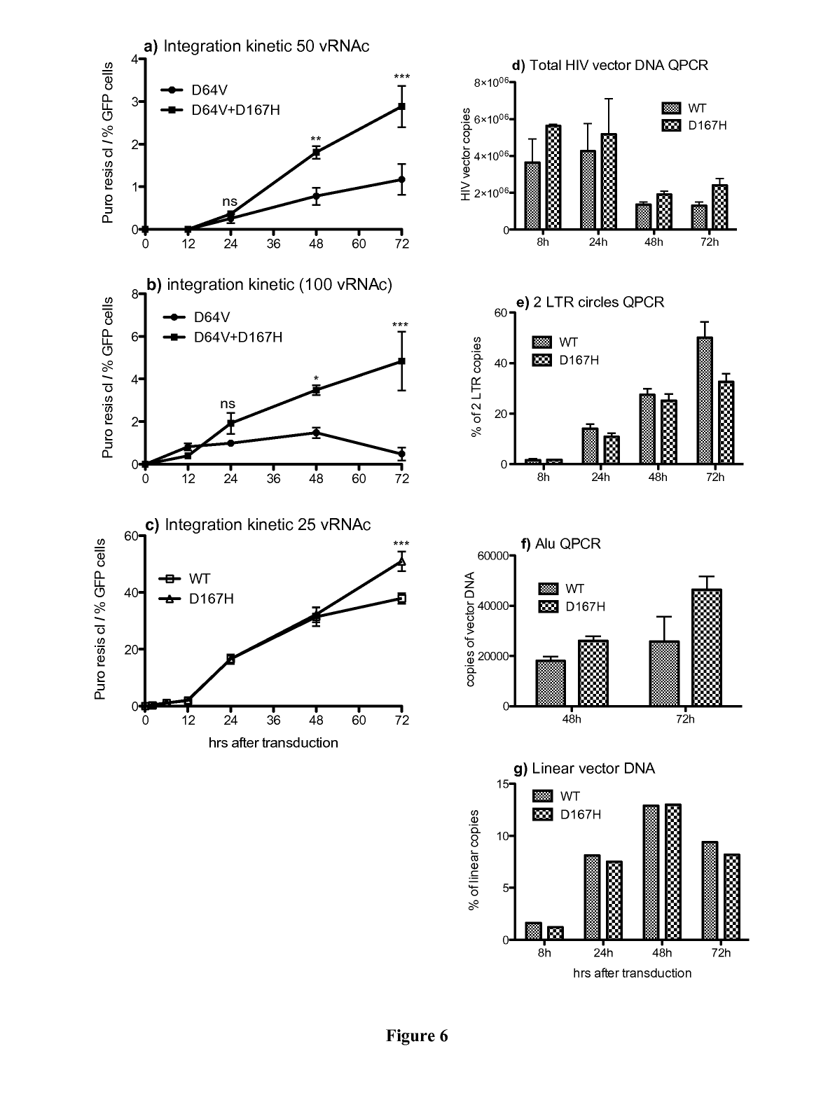

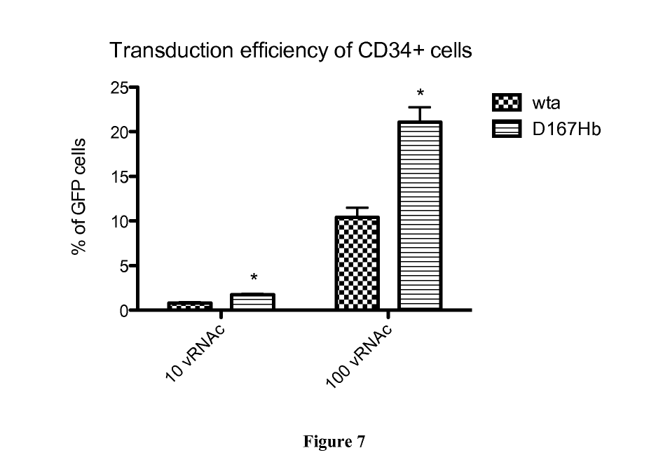

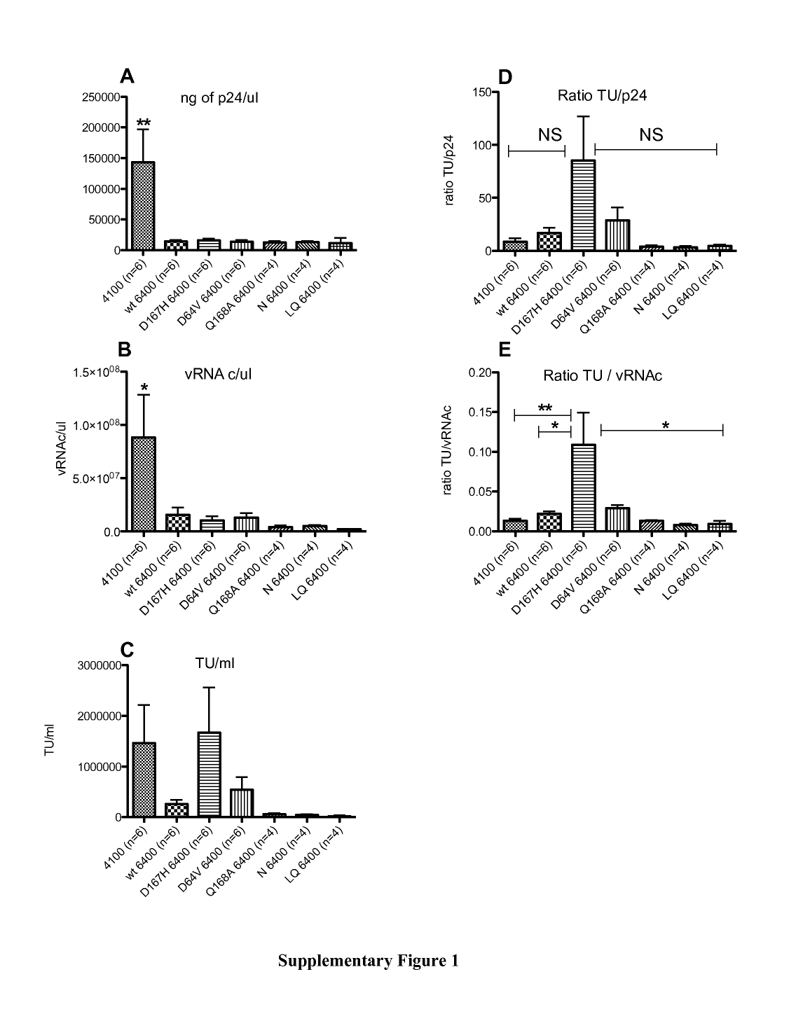

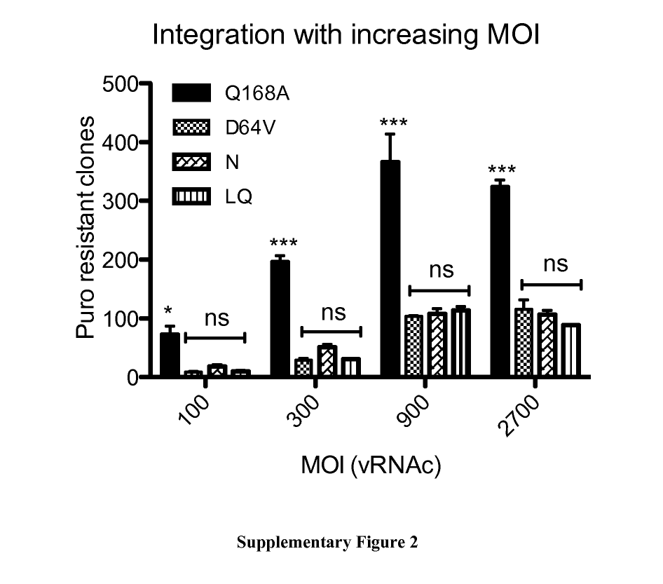

Supplementary FIG. 1

Titers of vector stocks assessed as measurements of p24/ul, vRNAc and TU/ul. Thirty-two stocks of vector were analysed to determine their content in viral capsid protein p24, in viral RNA copies (vRNAc) and transducing units per volume of supernatant. A) The content of p24/ul of supernatant is increased in stocks of a smaller size vector (4100 bp) as compared to all stocks of bigger vectors (6400 bp) whatever the IN carried (p<0.01), while no differences are retrieved between stocks of 6400 bp vectors. B) The content of vRNAc/ul is increased in stocks of a smaller size vector (4100 bp) as compared to all other 6400 bp vectors (p<0.05). There are no differences in p24 content between stocks of 6400 bp vectors whatever the IN carried. C) No differences in TU/ul are found between the different stocks of vectors. D) No statistical differences are found between groups when comparing values of the ratio TU/p24. E) Significant differences are measured when comparing the ratio TU/vRNAc, with higher values for D167H vectors as compared to any of all other vector types. Statistics, One way ANOVA and tuckey pot hoc test; (NS) p>0.05; (*) p<0.05; (**) p<0.01; (***) p<0.001.

Supplementary FIG. 2

Comparison of integration rate of different IDLY in 293T cells after transduction with increasing input of vector pTrip-CMV-GFP/PGK-PAC. The graph rates the number of puromycin resistant colonies counted 2 weeks after transduction with 100, 300, 900 or 2700 vRNAC/cell of vectors carrying either IN Q168A, D64V, N or LQ.

Experiments were done twice in duplicate. Non-transduced cells all died in puromycin medium. Statistics: two way ANOVA and Bonferroni posttest, NSp>0.05; (*) p<0.005; (**) p<0.01; (***) p<0.001.

### EXAMPLE

### Material & Methods

Plasmid Constructs and Vectors Production.

These 5 mutations were individually introduced in the p8.91 plasmid encoding the gag-pol genes of HIV-1. Plasmid p8.91 was obtained from (Zennou, V. et al. 2001 Nature Biotechnology). Plasmid D64V was obtained from (Nightingale, S J. 2006 Mol Ther). Plasmid p8.91_N was described earlier (Philippe et at 2006 PNAS USA), plamids p_8.91_LQ, p8.91_Q168A, p8.91_D167H and p8.91_D64V_D167H were constructed by inserting a SwaI-AflII synthesized DNA fragments (Genescript) from p8.91 containing a large part of the integrase and including either LQ, Q168A, D167H or D64V and D167H mutations. These fragments cloned in pUC57 were digested with SwaI-AflII restriction enzymes and cloned in p8.91 linearized with SwaI-AflII enzymes. Positive clones were sequenced.

Cells, Vectors and Transduction.

For vector production we used HEK293T cells (human embryonic kidney cell line, ATTC-CRL-11268) grown in DMEM (Life technologies) supplemented with 10% SVF and penicillin and streptomycin. Trancomplementation plasmids expressing structure and enzymes of HIV but deleted of accessory genes (p8.91), a plasmid expressing the vesicular stomatitis virus envelop glycoprotein (pVSV) were used to produce VSV pseudotyped pTrip PGK-Puro/CMV-Gfp self inactivating vector (SIN) vector particles as described in (Zennou, V. et al. 2001 Nature Biotechnology). Briefly, batches of HIV-1 derived vectors were produced by the transient cotransfection, of the various plasmids (p8.91, pVSV and pTrip PGK-Puro/CMV-Gfp in subconfluent HEK 293T cells cultured in 10 cm petri dishes, with the CaPO2 precipitate procedure. Each petri dish was incubated with 1 ml of precipitate, containing 13 ug of the packaging p8.91 plasmid, 3.75 ug of the Envelope plasmid and 13 ug of the vector plasmid) for 5 h then changed with fresh medium. Supernatant were harvested 48 h after transfection, filtered through a 0.45 um Stericup filters and centrifuged at 19,000 rpm (Beckman Coulter SW 32Ti rotor) for 1.5 h at 4° C. The supernatants were removed and the pellets were resuspended in 60 ul of 1% BSA in PBS, aliquoted and frozen at −80° C. until use.

Lentiviral Vector Titration

To determine recombinant particles content, we used the ELISA assay for the p24 antigen (Gentaur, France). The number of LV RNA copies were tittered by RTqPCR. The concentrated viral suspension (2 μl) was added to a 1.5 ml tube containing 353 μl DNase- and RNase-free water, 5 μl of DNAseI and 50 pg of RNaseA and incubated for 10 min at RT before adding 20 μl of RNasin (Promega). The mixture was incubated for 10 minutes at 37° C. and 10 μl of 25 mM EDTA for inactivation of DNase plus heating at 70° C. for 10 minutes. RNA was extracted with a Pure link RNA miniKit (Ambion) according to guidelines. At the end of the procedure, the purified RNA was eluted in 30 μl of the kit solution (in RNase-free tubes). Purified RNA (5 μl) was added to 96-well plate for reverse transcription with the Express Superscript mix and Express SYBR green ER supermix (Invitrogen). As a negative control, the RNA was added to the well without reverse transcriptase. PCR was carried out as follows: after reverse transcription for 5 minutes at 50° C., 2 minutes at 95° C. (denaturation), and 40 cycles of 15 seconds at 95° C. (denaturation), and one minute at 60° C. (amplification) with the primers Sense: TGTGTGCCCGTCTGTTGTGT (SEQ ID NO:2) Antisense: GAGTCCTGCGTCGAGAGAGC (SEQ ID NO:3). The number of RNA copies was obtained from a standard curve for known numbers of copies of DNA plasmid treated in the same way as the samples. The relative titer/ml was calculated as the number of RNA copies×dilution of vector preparation)/volume in ml.

Determination of Residual Integration

293T cells were plated (10⁵ cells/well in a 24 multiwell plate). The next day, cells were transduced with a M.O.I. of vectors (RNA copy/cell) allowing to transduce 30% of cells. After four days, cells were trypsinyzed and resuspended in 1 ml of medium and split in 2 petri dish (10 cm) with 100 and 900 ul respectively. Puromycin selection agent (Sigma) was added in the afternoon at a final concentration of 2 ug/ml and resistant clones were counted 10 day after. For kinetic assay transduction was performed in 293T cell in suspension. 2.5·10⁵ cell/tube were incubated for 1 h with the lentivirus vector before being plated in a 10 cm petri dish. One ml of puromicyn 10× was added in cells media at 2 h, 6 h, 10 h, 24, 48 or 72 h after transduction. Puromicyn resistant clone were count 10 day after. Non transduced cells were used as control.

Transduction Efficiency by Flow Cytometry

293T cells were plated (10⁵ cells/well in a 24 multiwell plate). The next day, cells were transduced with a number of RNA copy/cell allowing to transduce 30% of the cells (residual integration) or particular M.O.Is of vectors (transduction efficiency, kinetics of integration. Transduced cells were harvest at different times post transduction. (see above), trypsinised and fixed with formaldehyde 1% final. Then, cells were transferred to FACS tubes and Flow cytometry analysis was performed to determine the percentage of cells positive for GFP using the Cyflow Space device (Partec).

Statistics:

To evaluate statistical significance between vector titres differences, transduction efficiencies, integration rates or integration kinetics, data were analysed using standard statistical tests calculated by software graph pad prism 5. Employed tests are mentioned in figure legends.

Results:

Method to Titer Vector Stocks.

At least three methods are commonly used to establish the concentration of HIV vector particles in a supernatant [24]. One is functional and computes the number of successful particles leading to transgene expression in targeted cells (transducing units—TU/ml). Two other methods are physical, they dose viral capsid protein p24 with ELISA (ng of p24/ml) or viral RNA genome copies with qPCR (vRNAc/ml).

We first checked whether the mutations of IN (D64V; D167H; Q168A, LQ or N) affect any of these 3 parameters in HIV-vectors. We collected supernatants of 32 preps of 7 different types of vectors of which 2 carried a WT IN but had a different size, pTrip CMV-Gfp (4100 bp) or pTrip PGK-Puro/CMV-Gfp (6400 bp), and 5 carried either IN mutants, with the 6400 bp vector (FIG. 1). Comparison of variance between groups of p24 or vRNA concentrations in supernatants of different stocks of each vector types, revealed that these parameters were statistically increased in stocks of vectors with a smaller genome of 4100 bp as compared to vectors with longer genomes of 6400 bp (Supplementary FIGS. 1 A and B). However, no statistical differences in p24 and vRNAc concentration per ml of supernantants were observed between the different vectors of 6400 bp (Supplementary FIGS. 1 A and B). Then, although TU/ml was higher with stocks of WT 4100 bp and D167H 6400 bp vectors as compared to all other WT, D64V, Q168A, N and LQ, 6400 bp vectors, this difference wouldn't reach significance (FIG. S1 C). We then compared if the ratios (TU/ml)/(p24/ml) and (TU/ml)/(vRNA/ml) were different between groups of vectors. At difference from the ratio of (TU/ml)/(p24/ml) of D167H that was not statistically higher than that of all other vectors, that of (TU/ml)/(vRNA/ml) was significantly higher than that of all other vector types (D167H vs: WT 4100: p<0.01; D167H vs WT, 6400, D64V, Q168A, N or LQ: p<0.05), pointing at an improved transduction efficiency with this vector.

As TU/ml measurements are proportional to fitness and concentration of a vectors, any change altering the processing of particles during production or cell transduction may affect this titer. Though, as we needed to compare effects of class I and II IN mutations, a physical method for measuring particles concentration in each stock was required. To choose between ng of p24/ml and vRNAc/ml dosage to normalize and compare our vectors, we correlated variations of concentration of ng of p24/ml or vRNAc/ml with that of TU/ml of 32 different preps. We first correlated these variables for all stocks disregarding vector size or the carried IN. we observed that variation of concentration of vRNAc and p24 were both correlated to that of TU/ml of vector supernatants, although correlation between vRNA and TU/ml was more significant than that of p24 and TU/ml (FIG. 2). Analysis of correlations within each group of vectors, further confirmed this assessment as variations of vRNAc was statistically correlated to that of TU/ml for all types of vectors but one (LQ), while that of p24/ul reached significance for only 3 vector types (FIG. 2). Thus, as previously reported by others [25], this analysis confirms that measurement of vRNAc in vectors supernatants is more accurate than that of p24 to normalize and compare different kinds of HIV-derived vectors with regard to transduction efficiency. Consequently, in the following experiments we used vRNAc/ml to normalize amount of particles for cell transduction.

Transduction Efficiencies of IN Mutant Vectors.

Most of IN mutations compared here have been previously studied displaying different effects on enzyme biochemistry and virus processing (Table 1). They should thus have variable effects on transduction efficiency of vectors carrying these amino acid changes. Mutations D64V, N and Q168A were previously shown to induce a non-integrating phenotype in HIV vectors [15, 16, 26] but have never been directly compared. The 2 mutations D167H and LQ are studied in this context for the first time.

Using a double copy pTrip PGK-Puro/CMV-Gfp vector (FIG. 1A), we measured transgene expression at different time points after transduction of 6 types of HIV-1 derived vectors carrying either WT or mutated IN (D64V; D167H; Q168A, LQ or N) (FIG. 1B).

293T cells were incubated with low (10 vRNAc/cell) or higher (100 vRNAc/cell) amounts of WT or mutant IN vectors. Transduced cells were then analyzed with FACS at 4, 7, 14 and 21 days after transduction to define the percentage of GFP expressing cells and the mean fluorescence intensity (MFI) as a relative reference of vector copy number per cell. With 10 vRNAc/cell, we first observed that, as WT, D167H vector has an integrating phenotype allowing stable GFP expression over time in transduced cells (FIG. 3A). The 4 other mutants (D64V, Q168A, N and LQ) all displayed a non-integrating phenotype with GFP clearance over time (FIG. 3A). Most efficient integrating vector was the one carrying the D167H mutation, which allowed a 1.5 times higher transduction efficiency than wt vector; a significant increase noted at all time points of analysis (FIG. 3A). Both WT and D167H vectors allowed a peak of transduced cells a week after transduction (WT˜34% of GFP+ cells; D167H˜49%) followed by a slight decrease in number of GFP expressing cells after 2 weeks (WT˜21% of GFP+ cells; D167H˜31%) (FIG. 3A), possibly due to the presence of non-integrated 1 and 2 LTR circles at earlier time points, vanishing with cell divisions thereafter. Of the 4 IN mutants inducing a non-integrating phenotype, D64V efficiency of transduction was 2 to 6 times higher than that of other mutants. Mutant Q168A was the second most efficient; N and LQ were equivalent and the least efficient (FIG. 3B). Using 10 vRNAc/cell, we observed that MFI was slightly but significantly higher at 4 and 7 days in cells transduced with D167H as compared to WT, but that these values were equivalent for both vectors at last time points of analysis when the percentage of transduced cells was ≦30% (FIG. 3C). Vectors with non-integrating phenotype all permitted equivalent MFI that was about 5 times lower than that of integrating vectors, indicating a known decreased transcription by HIV episomes [21].

At higher dose of 100 vRNAc/cell, efficiencies of transduction of WT and D167H vectors appeared equivalent, as both allowed transduction of 100% of the cells (FIG. 3B). MFI, however, was about 1.5 times higher in cells transduced with D167H mutant vector as compared to WT, further indicating that D167H IN improves efficiency of cells transduction (FIG. 3D). Cells transduced with higher input of D167H vector (300 and 900 vRNAc/cell) also displayed an equivalent increased MFI as compared to WT vector indicating a non-saturating gain of function of the D167H mutant (not shown). Transduction efficiency of non-integrating mutants with 100 vRNAc/cell was much closer to that of WT and D167H with about 90% of transduced cells with D64V, 80% with Q168A and 50% with N or LQ at day 7 but quickly decreasing thereafter (FIG. 3c). MFI of non-integrating vectors peaked at day 4 after transduction and remained stable thereafter. At Day 4, cells transduced with mutants D64V, Q168A, N and LQ were about 4 and 6 times less fluorescent than WT and D167H respectively. At greater doses of vectors of 300 and 900 vRNAc/cell, the 4 non-integrating vectors allowed transduction of 100% of the cells at day 7 (not shown).

Thus D167H mutant vector has an integrating phenotype and appears improved for transducing 293T cells. Of the 4 non-integrating vectors compared, the one carrying the D64V mutation displays the best efficiency of transduction.

We next evaluated the rate of integration allowed by the different vectors with wt and mutated integrases.

Residual Integration of LV with Different IN Mutations.

Some previously characterized effects of the different mutations of IN that we compare in this study are recapitulated in a table (Table 1). Mutation D64V abolishes catalytic activity of IN [27] while mutations Q168A and N respectively impair interaction with cellular factors p75/LEDGF and karyopherin TNPO3, reducing viral cDNA integration rate [7, 8, 26]. Substitutions named LQ affect IN dimerization, interaction with p75/LEDGF and nuclear interaction between IN and a cellular host factor implicated in integration [19, 28], seemingly karyopherin-al [29, 30]. Substitutions at position D167A/K/C have ambiguous effects on HIV IN enzymatic activity [18, 23, 31], and, depending on the nature of the substitution, reduce IN interaction with LEDGF/p75 [18], affinity for viral DNA substrate, HIV replication [18, 31] and worsen a non-replicating phenotype of HIV-1 when associated to mutation R166A [31], but mutation D167H is studied for the first time. Thus, as the integrating and non-integrating phenotypes of these mutants are induced through different effects on IN, their respective rate of integration might also be different.

To measure the rate of integration of vectors WT, D64V, Q168A, N, LQ and D167H we thought to compare integration events in populations of cells containing the same amount of nuclear proviral DNA. A statistical model based on Poisson distribution indicates that when at most 30% of a cell population is infected/transduced, each targeted cells contain 1 copy of virus [5], verified in practice for lentiviral vectors [32].

Thus for each stock of vector we first defined the functional titer in TU/ml. We then used appropriate amount of particles to allow transduction of 30% of 293T cells and verified it with FACS after 4 days. We observed that when 30% of 293T cells had been transduced with either integrating (WT and D167H) or non-integrating (D64V, Q168A, N, LQ) vectors, their MFI were respectively equivalent (not shown), indicating that they all contained a similar copy number of vector per cell. Four days after transduction, puromycin was added in the culture medium to select cells having integrated a copy of pTRIP-CMV-Gfp/PGK-Puro vector.

Twelve days after transduction, we counted the colonies of resistant cells that had appeared in the plates. By comparing all groups, we observed that WT and D167H vectors gave a number of colonies that was significantly higher than non-integrating vector D64V, Q168A, N and LQ (p<0.001) (FIGS. 4 A and B). In these conditions, we also observed that mutant D167H produced about 1.5 times more colonies than a vector with WT IN (p<0.001) (FIG. 4 A). Mutant D64V was the least integrative giving about 700 times less colonies than WT and about 5 times less colonies than mutants N and LQ (p<0.001). Mutant Q168A had an intermediary phenotype and was about 15 times less integrative than WT and 10 to 50 times more than mutants LQ and N or D64V respectively (p<0.001).

Thus of all vectors compared, that containing the D167H mutant IN is the most integrating while that containing the D64V mutation is the one allowing less integration events. We next evaluate if the D167H substitution modifies the phenotype of a D64V vector.

IN with D64V/D167H Substitutions Allows More Integration Event than that with D64V.

Previous experiments strongly suggest that D167H mutation potentiate viral vectors integration. To further study if D167H mutation acts potentiating catalytic activity of IN or through any other mechanism, we associated it to the D64V mutation. We thus introduced the 2 mutations in the IN sequence of the transcomplementation plasmid p8.91 (p8.91-D64V/Q167H). Particles carrying the single D64V or the double D64V/Q167H mutant IN were used to transduce 293T cells with 100 vRNAc of each vector. Both vectors displayed equivalent transduction efficiency (FIG. 5A) and a similar non-integrating phenotype with loss of GFP expression through cells division (FIG. 5B). Surprisingly, when we measured integration, by selecting pruomycin resistance starting at day 4 after transduction, we observed that the double mutant D64V/Q167H led to a significant increased number of colonies (1.5 times more) as compared to the single mutant D64V (FIG. 5C). This either suggests that the D64V mutant conserves a residual enzymatic activity that is further potentiated by the mutation D167H or that the D167H mutation introduces a qualitative change in vectors that improves PIC stability and IN independent integration in cellular chromatin.

Kinetics of Integration of Vectors Carrying the D167H Mutation.

We reasoned that the D167H might modify the processing of the vector improving its stability and thus change its kinetics of integration. Thus, to further understand the mechanism of integration of particles bearing this mutation, we transduced 293T cells with vectors WT, D167H, D64V or D64V+D167H and correlated efficiency of transduction (GFP expression) to integration (stable puromycin expression) in time (0, 6, 12, 24, 48 and 72 hrs). For each point of analysis, cells were split in 2 wells just after transduction, for measuring either, GFP expression and integration of the different vectors. GFP was measured with FACS at mentioned time points after transduction, while integration was extrapolated as the number of puromycin resistant colonies appearing with selection drug added at mentioned times after transduction. At each time point, efficiency of integration was rated as the number of puromycin resistant colonies divided by percentage of cells expressing GFP. When comparing integration of D64V and D64V+D167H in 293T cells transduced with 50 or 100 vRNAc of vectors, we observed that the more we moved further in time post-transduction, the higher the difference between the two vectors (FIGS. 6A and B). Integration of D64V vectors mainly occurred between 0 and 48 hrs after transduction then seemingly reaching a plateau. Integration of D64V+D167H, instead, kept increasing linearly until the last time point analyzed at 72 hrs after transduction. The kinetics of integration of integrating vectors was also different between WT and D167H vectors. The former tended to reach a plateau of integration at 48 hrs after transduction, as reported before, while the mutant kept integrating between 48 and 72 hrs after transduction (FIG. 6 C).

These results suggest that IN D167H substitution modify the kinetics of integration of HIV vectors through a mechanism increasing viral genome stability.

LV with D167H Substitution is Improved CD34+ Cells Transduction.

Retroviral vectors have been used successfully to genetically modify hematopoietic progenitors and treat congenital immunodeficiencies. We wondered if HIV vectors bearing a D167H IN were improved compared to WT for transducing CD34+ cells. CD34+ primary human cells in culture were incubated with equal vRNAc of vectors D167H and WT. After 4 days, cells were analyzed by FACS to assess the percentage of transduction. With low and high dose of vectors, we observed that particles carrying the D167H IN allowed transducing about 2 times more CD34+ cells as compared to WT particles (FIG. 7).

Integration Profile of LV with Different IN Mutations.

To further compare the properties of mutants and WT vectors, we assessed their respective pattern of integration in 293T cells with non-restrictive LAM-PCR. Cells were transduced with increasing input of vectors (100, 300, 900 and 2800 vRNAc/cell) and integration events were selected by adding puromycin at day 4 after transduction. After 10 days, colonies were counted, pooled and further cultured before genomic DNA purification. To analyze the pattern of integration of vectors WT and D167H we used the DNA of colonies obtained with the lower vector input (100 vRNAc/cell). For IDLV, which result in fewer integration events, we mixed the DNA of all colonies obtained with the different doses of vectors (Supplementary FIG. 2). Non-restrictive LAM-PCR was then performed on DNA of cells transduced with each vector and PCR products were sequenced using Illumina MiSeq technology, and flanking genomic sequences were characterized with bioinformatical data mining. As expected, the number of IS obtained from samples transduced with vectors carrying mutations impairing integration is clearly lower than from samples transduced with a integrating vectors. Vector carrying IN D167H, instead, gave approximately 1.4 more integration sites (IS) than WT vector, which again points at an increased integration rate of D167H vector as compared to that carrying a WT IN. The lower frequency of IS in cells transduced with IDLV was most prominent for D64V, N, and LQ mutations in comparison to data obtained with vectors carrying mutation Q168A, a result that is also in line with the higher rate of residual integration observed with this vector (FIG. 4) and the higher number of clones obtained after transducing 293T cells (Supplementary FIG. 2). Previous analysis of integration patterns of lentiviral vectors showed that integrated genomes of vectors carrying a D64V IN suffer about 10 times more deletions within LTRs than that of vector genomes integrated by a WT IN. Here we also observed that mutant IN impairing integration produced increased number of LTR deletions (Q168A, 1.8%; LQ, 16.8%; D64V, 21.6%; N, 44.7%), while D167H and WT IN displayed the same low frequency of deletion (0.3%). The differences in frequency of LTR deletions observed with the different vectors suggest that integration proceeds through mechanisms with variable involvement of IN or IN-interacting factors. We further characterized the localization of IS of our vectors within chromosomes and gene coding regions. Previous studies showed that HIV and derived vectors integrate preferentially within transcribed genes (70%) and a reduced frequency of IS within gene coding regions for D64V vectors (Gabriel et al., 2011, 2009; Matrai et al., 2011; Paruzynski et al., 2010; Schmidt et al., 2007) or in cells depleted in LEDGF or TNPO3. Here, we also observed that vectors carrying Q168A, D64V, LQ or N mutations reduced the frequency of HIV vector integration within or around genes as compared to WT and D167H vectors. The reduced frequency of integration within genes-dense as compared to WT and D167H (70% of integration in gene coding regions) regions was variable depending on vectors; It was moderate for the mutant Q168A (˜65%), but more prominent for the mutant N (˜45%), as compared to D64V and LQ (˜50%). This again points at different mechanisms of integration with these different vectors.

Furthermore, the occurrence and frequency of “common integration sites” (CIS), regions in the genome where multiple vector integrations occurred, was determined. CIS detection enables to uncover integration hotspots and are an indicator for clonal skewing in gene therapy protocols (Ott et al., 2006). We employed the following definition for CIS determination: 2nd order CIS: 2 IS in 30 kb; 3rd order CIS: 3 IS in 50 kb; 4^{th} order CIS: 4 IS in 100 kb; ≧5^{th} order CIS: 5 or more IS in 200 kb). CIS analysis was performed separately for individual vector data set.

The frequency of CIS is in line with the number of IS which could be retrieved for the individual data set. The highest CIS order observed is CIS of 27^{th} order in the D167H data set occurring on chromosome 8. Also in the WT data set, the highest CIS of 15^{th} order was identified in the same genomic region of chromosome 8. We can see that the mean of highest order of CIS is 11.4 for vectors with WT IN but is 1.6 times higher for vectors D167H (18.6) and 2 to 6 times lower for vectors Q168A and other IDLVs. These data also reveal that in 293T cells, as in human CD34+ cells, typical lentiviral integration hotspot regions like PACS1 are retrieved as CIS of high order, but also that particular gene regions, which have not been previously described as lentiviral hotspot, are preferentially targeted, like CIS on chromosome 8 or 16.

TABLE 1

| | | LEDGF | TNPO3 | | | DNA | Nuc. | | | C |
|---|---|---|---|---|---|---|---|---|---|---|
| Δaa | RT | binding | binding | Oligom. | 3′ p | st | tr. | Integrat. | Infect | I/II |
| D64N | ↓[23] | →[35] | ND | →[23] | ↓↓↓ | ↓↓↓ | ND | ND | ↓↓↓[35] | I |
| D64E | →[33] | | | | [23] | [23] | →[36] | ↓↓[36] | ↓↓↓[33] | I |
| D64V | →[34] | | | | | | ND | ↓↓↓[33] | ↓↓[34] | I |
| | | | | | | | →[34] | ↓↓↓[34] | | |
| D167K | ↓[23] | ↓[35] | ND | →[23] | ↓[23] | ↑[23] | ND | ND | ↑[35] | II |
| D167A | | ↓[35] | ND | ND | ND | ND | ND | ↓[31] | ↓↓[35] | II |
| Q168A | ↓[23] | ↓[35] | ND | ↓↓[23] | →[23] | ↑[23] | →[37] | ↓↓[37] | ↓↓ | II |
| | | ↓↓[37] | | | | | | | [35, 37] | |
| K186Q | ↓[23] | ↓[23] | ND | ↓↓[23] | ↓[23] | ↓↓[23] | ↓[19] | ↓↓[19] | ↓↓[19] | II |
| | | | | ↓[19] | | | | | | |
| Q214 & 216L | ↓[23] | ↓↓↓[23] | ND | →[23] | ↓↓[23] | ↓↓[23] | ↓[19] | ↓↓[19] | ↓↓[19] | II |
| | | | | ↓[19] | | | | | | |
| K186Q + | ND | ND | ND | ↓↓[19] | ND | ND | ↓[19] | ↓↓[19] | ↓↓[19] | II |
| Q214 & 216L | | | | | | | | | | |
| RRK262/ | ↓[38] | → | ↓↓ | → | ND | ND | →[19] | ↓↓[19] | ↓↓[19] | II |
| 64AAH | | [7, 8] | [7, 8] | [7, 19] | | | | | | |

The effect of different mutations of IN compared in this study that were previously studied in biochemical or virological studies are recapitulated in this table (not exhaustive). Note that the type of amino acid substitution may have different effect on IN kinetic. (RT) reversetranscription, (Oligom) oligomerization of IN, (3′ p) 3′ processing of HIV proviral extremities, (DNA st) DNA strand transfer, (Nuc. tr) nuclear translocation, (Integrat) integration, (infect) infectiosity, (Cl/II) class I or II mutation of IN. → normal, ↑ increased, ↓ decreased (between 50 and 99%), ↓↓ decreased (between 1 and 49%), ↓↓↓ abolished.

### REFERENCES

Throughout this application, various references describe the state of the art to which this invention pertains. The disclosures of these references are hereby incorporated by reference into the present disclosure.
1. Naldini, L, et al. (1996). In vivo gene delivery and stable transduction of nondividing cells by a lentiviral vector. Science 272: 263-267.
2. Matrai, J, Chuah, M K, and VandenDriessche, T (2010). Recent advances in lentiviral vector development and applications. Mol Ther 18: 477-490.
3. Arhel, N (2010). Revisiting HIV-1 uncoating. Retrovirology 7: 96.
4. Hare, S, and Cherepanov, P (2009). The Interaction Between Lentiviral Integrase and LEDGF: Structural and Functional Insights. Viruses 1: 780-801.
5. Fields, B N, Knipe, D M, and Howley, P M (2007). Fields virology, Wolters Kluwer Health/Lippincott Williams & Wilkins: Philadelphia.
6. Craigie, R, and Bushman, F D (2012). HIV DNA Integration. Cold Spring Harb Perspect Med 2: a006890.
7. De Houwer, S, et al. (2012). Identification of residues in the C-terminal domain of HIV-1 integrase that mediate binding to the transportin-SR2 protein. J Biol Chem 287: 34059-34068.
8. Lame, R, et al. (2012). Interaction of the HIV-1 intasome with transportin 3 protein (TNPO3 or TRN-SR2). J Biol Chem 287: 34044-34058.
9. Ocwieja, K E, et al. (2011). HIV integration targeting: a pathway involving Transportin-3 and the nuclear pore protein RanBP2. PLoS Pathog 7: e1001313.
10. Schroder, A R, Shinn, P, Chen, H, Berry, C, Ecker, J R, and Bushman, F (2002). HIV-1 integration in the human genome favors active genes and local hotspots. Cell 110: 521-529.
11. Cavazzana-Calvo, M, et al. (2010). Transfusion independence and HMGA2 activation after gene therapy of human beta-thalassaemia. Nature 467: 318-322.
12. Lai, Z, Han, I, Park, M, and Brady, R O (2002). Design of an HIV-1 lentiviral-based gene-trap vector to detect developmentally regulated genes in mammalian cells. Proc Natl Acad Sci USA 99: 3651-3656.
13. Meehan, A M, et al. (2009). LEDGF/p75 proteins with alternative chromatin tethers are functional HIV-1 cofactors. PLoS Pathog 5: e1000522.
14. Schenkwein, D, Turkki, V, Ahlroth, M K, Timonen, O, Airenne, K J, and Yla-Herttuala, S (2013). rDNA-directed integration by an HIV-1 integrase—I-PpoI fusion protein. Nucleic Acids Res 41: e61.
15. Philippe, S, et al. (2006). Lentiviral vectors with a defective integrase allow efficient and sustained transgene expression in vitro and in vivo. Proc Natl Acad Sci USA 103: 17684-17689.
16. Yanez-Munoz, R J, et al. (2006). Effective gene therapy with nonintegrating lentiviral vectors. Nat Med 12: 348-353.
17. Iglesias, C, et al. (2011). Residual HIV-1 DNA Flap-independent nuclear import of cPPT/CTS double mutant viruses does not support spreading infection. Retrovirology 8: 92.
18. Busschots, K, et al. (2007). Identification of the LEDGF/p75 binding site in HIV-1 integrase. J Mol Biol 365: 1480-1492.
19. Petit, C, Schwartz, O, and Mammano, F (2000). The karyophilic properties of human immunodeficiency virus type 1 integrase are not required for nuclear import of proviral DNA. J Virol 74: 7119-7126.
20. Wanisch, K, and Yanez-Munoz, R J (2009). Integration-deficient lentiviral vectors: a slow coming of age. Mol Ther 17: 1316-1332.
21. Sarkis, C, Philippe, S, Mallet, J, and Serguera, C (2008). Non-integrating lentiviral vectors. Curr Gene Ther 8: 430-437.
22. Engelman, A (1999). In vivo analysis of retroviral integrase structure and function. Adv Virus Res 52: 411-426.
23. Li, X, Koh, Y, and Engelman, A (2012). Correlation of recombinant integrase activity and functional preintegration complex formation during acute infection by replication-defective integrase mutant human immunodeficiency virus. J Virol 86: 3861-3879.
24. Kutner, R H, Zhang, X Y, and Reiser, J (2009). Production, concentration and titration of pseudotyped HIV-1-based lentiviral vectors. Nat Protoc 4: 495-505.
25. Geraerts, M, Willems, S, Baekelandt, V, Debyser, Z, and Gijsbers, R (2006). Comparison of lentiviral vector titration methods. BMC Biotechnol 6: 34.
26. Vandekerckhove, L, et al. (2006). Transient and stable knockdown of the integrase cofactor LEDGF/p75 reveals its role in the replication cycle of human immunodeficiency virus. J Virol 80: 1886-1896.
27. Li, X, Krishnan, L, Cherepanov, P, and Engelman, A (2011). Structural biology of retroviral DNA integration. Virology 411: 194-205.
28. Lu, R, Limon, A, Devroe, E, Silver, P A, Cherepanov, P, and Engelman, A (2004). Class II integrase mutants with changes in putative nuclear localization signals are primarily blocked at a postnuclear entry step of human immunodeficiency virus type 1 replication. J Virol 78: 12735-12746.
29. Gallay, P, Hope, T, Chin, D, and Trono, D (1997). HIV-1 infection of nondividing cells through the recognition of integrase by the importin/karyopherin pathway. Proc Natl Acad Sci USA 94: 9825-9830.
30. Hearps, A C, and Jans, D A (2006). HIV-1 integrase is capable of targeting DNA to the nucleus via an importin alpha/beta-dependent mechanism. Biochem J 398: 475-484.
31. Wiskerchen, M, and Muesing, M A (1995). Human immunodeficiency virus type 1 integrase: effects of mutations on viral ability to integrate, direct viral gene expression from unintegrated viral DNA templates, and sustain viral propagation in primary cells. J Virol 69: 376-386.
32. Charrier, S, et al. (2011). Quantification of lentiviral vector copy numbers in individual hematopoietic colony-forming cells shows vector dose-dependent effects on the frequency and level of transduction. Gene Ther 18: 479-487.
33. Masuda, T, Planelles, V, Krogstad, P, and Chen, I S (1995). Genetic analysis of human immunodeficiency virus type 1 integrase and the U3 att site: unusual phenotype of mutants in the zinc finger-like domain. J Virol 69: 6687-6696.
34. Leavitt, A D, Robles, G, Alesandro, N, and Varmus, H E (1996). Human immunodeficiency virus type 1 integrase mutants retain in vitro integrase activity yet fail to integrate viral DNA efficiently during infection. J Virol 70: 721-728.
35. Rahman, S, Lu, R, Vandegraaff, N, Cherepanov, P, and Engelman, A (2007). Structure-based mutagenesis of the integrase-LEDGF/p75 interface uncouples a strict correlation between in vitro protein binding and HIV-1 fitness. Virology 357: 79-90.
36. Ao, Z, Fowke, K R, Cohen, E A, and Yao, X (2005). Contribution of the C-terminal tri-lysine regions of human immunodeficiency virus type 1 integrase for efficient reverse transcription and viral DNA nuclear import. Retrovirology 2: 62.
37. Emiliani, S, et al. (2005). Integrase mutants defective for interaction with LEDGF/p75 are impaired in chromosome tethering and HIV-1 replication. J Biol Chem 280: 25517-25523.
38. Lu, R, Ghory, H Z, and Engelman, A (2005). Genetic analyses of conserved residues in the carboxyl-terminal domain of human immunodeficiency virus type 1 integrase. J Virol 79: 10356-10368.

1. A lentiviral vector expressing an integrase protein comprising at least one point mutation that is a substitution of an aspartic acid residue at position 167 by an amino acid selected from the group consisting of histidine, arginine and lysine.

2. The lentiviral vector of claim 1 wherein the amino acid is histidine.

3. The lentiviral vector of claim 1 which is selected from the group consisting of HIV-1, HIV-2, Simian Immunodeficiency Virus (SIV), Feline Immunodeficiency Virus (FIV), Equine Infectious Anemia Virus (EIAV), Bovine Immunodeficiency Virus (BIV), Visna and Caprine Arthritis Encephalitis Virus (CAEV).

4. The lentiviral vector of claim 2 which is a HIV-1 vector.

5. The lentiviral vector of claim 1 which is a non-replicative and non-integrative recombinant lentivirus.

6. The lentiviral vector of claim 1 wherein the expressed integrase protein is devoid of a capacity for integration of a lentiviral genome into a genome of host cells.

7. The lentiviral vector of claim 6 wherein the expressed integrase protein comprises at least one further mutation selected from the group consisting of H12N, H12C, H16C, H16V, S81R, D41A, K42A, H51A, Q53C, D55V, D64E, D64V, E69A, K71A, E85A, E87A, D116N, D116I, D116A, N120G, N120I, N120E, E152G, E152A, D35E, K156E, K156A, E157A, K159E, K159A, K160A, R166A, E170A, H171A, K173A, K186Q, K186T, K188T, E198A, R199C, R199T, R199A, D202A, K211A, Q214L, Q216L, Q221 L, W235F, W235E, K236S, K236A, K246A, G247W, D253A, R262A, R263A and K264H.

8. The lentiviral vector of claim 6 wherein the expressed integrase protein comprises at least one further mutation which is replacement of an amino acid sequence RRK at positions 262 to 264 by an amino acid sequence AAH.

9. The lentiviral vector of claim 1 wherein an envelope protein of the lentiviral vector is a VSV-G protein.

10. The lentiviral vector of claim 1 having a recombinant genome comprising, between 5 ‘and 3’ lentiviral LTR sequences, a psi lentiviral packaging sequence, a nuclear export element RNA, and a transgene.

11. A vector genome for the preparation of a lentiviral vector, wherein the vector genome encodes a lentiviral vector expressing an integrase protein comprising at least one point mutation that is a substitution of an aspartic acid residue at position 167 by an amino acid selected from the group consisting of histidine, arginine and lysine.

12. A method for obtaining a lentiviral vector expressing an integrase protein comprising at least one point mutation that is a substitution of an aspartic acid residue at position 167 by an amino acid selected from the group consisting of histidine, arginine and lysine, comprising
transfecting in vitro a permissive cell with a plasmid containing a lentiviral vector genome encoding the lentiviral vector, and at least one other plasmid providing, in trans, gag, pol and env sequences encoding GAG, POL and envelope polypeptides, or encoding a portion of the GAG, POL and envelope polypeptides sufficient to enable formation of retroviral particles, and
harvesting the lentiviral vector.

13. A method for expressing a transgene in a mammalian cell, comprising
transducing the mammalian cell with a lentiviral vector comprising the transgene, wherein the lentiviral vector expresses an integrase protein comprising at least one point mutation that is a substitution of an aspartic acid residue at position 167 by an amino acid selected from the group consisting of histidine, arginine and lysine.

14. The method according to claim 13 wherein the mammalian cell does not divide or is a cell that is refractory to other methods of transfection or transduction.

15. The method according to claim 14 wherein the cell is selected from the group consisting of liver cells, muscle cells, brain cells, kidney cells, retinal cells, and hematopoietic cells.

16. A method of performing gene therapy in a subject in need thereof, comprising
delivering a gene of interest to the subject by administering to the subject a lentiviral vector that expresses the gene of interest, wherein the lentiviral vector expresses an integrase protein comprising at least one point mutation that is a substitution of an aspartic acid residue at position 167 by an amino acid selected from the group consisting of histidine, arginine and lysine.

17. A method for eliciting an immune response to an antigenic polypeptide in a subject in need thereof, comprising
administering to the subject a lentiviral vector comprising a transgene encoding the antigenic polypeptide, wherein the lentiviral vector expresses an integrase protein comprising at least one point mutation that is a substitution of an aspartic acid residue at position 167 by an amino acid selected from the group consisting of histidine, arginine and lysine, and wherein the antigenic polypeptide is expressed from said transgene in the subject so as to elicit an immune response to the antigenic polypeptide.

18. A method for the treatment of a disease selected from the group consisting of cancer, autoimmune disease, ocular disorders, blood disorders, neurological disorders, lung disorders, and infectious diseases in a subject in need thereof comprising administering to the subject a therapeutically effective amount of a lentiviral vector comprising a transgene that expresses an agent that treats the disease, wherein the lentiviral vector expresses an integrase protein comprising at least one point mutation that is a substitution of an aspartic acid residue at position 167 by an amino acid selected from the group consisting of histidine, arginine and lysine.

19. The lentiviral vector of claim 10 further comprising a promoter and/or a sequence favoring nuclear import of RNA.
